(19) United States
(12) Patent Application Publication
(10) Pub. No.: US 2025/0076280 A1
(43) Pub. Date: Mar. 6, 2025

(54) RAPID SCREENING FOR ELEVATED BILE ACIDS USING WHOLE BLOOD

(71) Applicant: The University of Toledo, Toledo, OH (US)

(72) Inventors: Matam Vijay-Kumar, Toledo, OH (US); Beng San Yeoh, Toledo, OH (US); Ahmed Abokor, Toledo, OH (US)

(73) Assignee: The University of Toledo, Toledo, OH (US)

(21) Appl. No.: 18/844,313

(22) PCT Filed: Mar. 8, 2023
(86) PCT No.: PCT/US2023/014778

§ 371 Date: Sep. 5, 2024

Related U.S. Application Data
(60) Provisional application No. 63/317,703, filed on Mar. 8, 2022.

(51) Int. Cl. G01N 33/49; G01N 21/29

(52) U.S. Cl. CPC G01N 33/49; G01N 21/293; G01N 2800/22

(57) ABSTRACT

Methods and assays for diagnosing cholemia are described. The methods and assays utilize the discovery that cholemic blood is resistant to osmotic lysis.

### RELATED APPLICATIONS

This application claims priority to U.S. Provisional Application No. 63/317,703 filed under 35 U.S.C. § 111(b) on Mar. 8, 2022, the disclosure of which is incorporated herein by reference in its entirety.

### STATEMENT REGARDING FEDERALLY SPONSORED RESEARCH

This invention was made with government support under Grant Number 5R01CA219144 awarded by the National Institutes of Health. The government has certain rights in this invention.

### BACKGROUND

Bile acids are amphipathic molecules synthesized in the liver and transported to the gallbladder at high concentration via bile. Bile acids are hydrophobic, detergent-like molecules that can exert toxic effects on cells, though they are required for digestion and absorption of dietary fats and fat-soluble vitamins. An essential function of bile acids is reflected in their detergent-like property to emulsify and assimilate lipid-derived nutrients. However, bile acids' capacity to breakdown lipids comes as a cytotoxic double-edged sword because they can permeate cell membranes and cause irreversible damage, such as eryptosis in red blood cells (RBCs). It has been found that bile acid-induced RBC hemolysis is from altering the cholesterol to phospholipid ratio, where the latter is severely depleted and restoration of the former lipid can protect erythrocyte membranes from bile acid toxicity. These in vitro findings were from RBCs isolated from healthy mammals and those RBCs were exogenously introduced to commercially available bile acids at very high concentration. There has been limited in vivo investigation to determine RBC integrity in mammals who exhibit high bile acid levels past the physiological threshold.

Cholemia and cholestasis are pathological conditions that feature excess circulating bile acids. A symptomatic feature of cholestasis, which is absent in asymptomatic cholemia, is jaundice caused by an overload of bilirubin, a byproduct of heme degradation and another component of bile. Both liver diseases can be caused from either functional impairment of bile-secreting parenchymal cells in the liver or physical obstruction within the bile ducts, resulting in an impediment of bile egress from liver to gallbladder. It has been found that jaundiced humans exhibit resistance to RBC hemolysis. However, it has been unclear what the contributing factors for this protection against osmotic shock are, since both bile acids and bilirubin induce hemolysis.

There are many causes from which humans suffer from cholemia or cholestasis, including genetic syndromes, diet, hormones, infections, drugs, inflammation, and environmental factors. Inflammation, scarring, or physical obstruction of the bile ducts lead to cholestasis. Spillover of excess bile into systemic circulation leads to elevated quantities of bilirubin and bile acids in the blood (jaundice and cholemia, respectively). Cholemia involves high bile acids with low bilirubin, often causing no symptoms, whereas jaundice involve high bile acids and high bilirubin. In patients having cholemia, bilirubin and liver function testing may give false negative readings, and follow-up bile acid testing is not typically conducted. While jaundice has been extensively studied, cholemia is largely asymptomatic with no existing methodology, and while cholemia is a blood condition, the effects of chronically elevated bile acids on red blood cell physiology are not fully known.

Currently, the gold standard for liver function tests is an assay of serum ALT (aka SGPT), and measuring serum bilirubin. It is rare for total bile acids to be measured. However, large molecular weight biomarkers for cholestasis, such as alkaline phosphatase (common in liver function tests), are not always reliable. As a result, commonly used liver function tests do not actually measure liver function, but, rather, merely detect liver injury. There is a need in the art for new and improved methods and assays for detecting cholemia.

### SUMMARY

Provided is a method for diagnosing cholemia, the method comprising obtaining a blood sample from a subject; adding a salt to the blood sample to form a solution; and comparing an amount of hemolysis that takes place in the solution to a reference amount of hemolysis in a normal blood sample in a reference solution with the salt to determine whether the subject has cholemia.

In certain embodiments, the method comprises comparing an opacity of the solution to a reference opacity from a normal blood sample in a reference solution with the salt, wherein the opacity of the solution being greater than the reference opacity indicates that the subject has cholemia. In particular embodiments, the salt comprises NaCl. In particular embodiments, the salt is present at a concentration ranging from about 0.30% by weight to about 0.48% by weight. In particular embodiments, the salt comprises KCl. In particular embodiments, the salt is present at a concentration ranging from about 0.55% by weight to about 0.65% by weight. In particular embodiments, the salt is present at a concentration ranging from about 0.45% by weight to about 0.5% by weight.

In certain embodiments, the method comprises comparing a color of a supernatant from the solution following centrifugation to a reference color from a normal blood sample in a reference solution with the salt following centrifugation, wherein the color of the supernatant being less red than the reference color indicates that the subject has cholemia. In particular embodiments, the method further comprises incubating the solution for a period of time of from about 15 minutse to about 30 minutes before centrifuging the solution. In particular embodiments, the salt comprises NaCl. In particular embodiments, the salt is present at a concentration ranging from about 0.30% by weight to about 0.48% by weight. In particular embodiments, the salt comprises KCl. In particular embodiments, the salt is present at a concentration ranging from about 0.55% by weight to about 0.65% by weight. In particular embodiments, the salt is present at a concentration ranging from about 0.45% by weight to about 0.5% by weight.

In certain embodiments, the method comprises comparing a pellet size of the solution to a reference pellet size of a normal blood sample solution with the salt.

In certain embodiments, the subject is a human.

Further provided is a method for evaluating resistance to lysis, the method comprising either (a) observing a difference in opacity between a first blood sample and a second blood sample, and concluding from the difference in opacity that one of the first blood sample or the second blood sample is more resistant to lysis; or (b) observing a difference in color between a first supernatant from a first blood sample and a second supernatant from a second blood sample, and concluding from the difference in color that one of the first blood sample or the second blood sample is more resistant to lysis.

Provided is a method for diagnosing cholemia, the method comprising obtaining a blood sample from a subject; adding a salt to the blood sample to form a solution; and comparing an opacity of the solution to a reference opacity of a normal blood sample in a reference solution with the salt to determine whether the subject has cholemia, wherein the opacity of the solution being greater than the reference opacity indicates that the subject has cholemia.

In certain embodiments, the salt comprises NaCl. In particular embodiments, the salt is present at a concentration ranging from about 0.30% by weight to about 0.48% by weight.

In certain embodiments, the salt comprises KCl. In particular embodiments, the salt is present at a concentration ranging from abou 0.55% by weight to about 0.65% by weight. In particular embodiments, the salt is present at a concentration ranging from about 0.45% by weight to about 0.5% by weight.

In certain embodiments, the subject is a human.

Further provided is a method for diagnosing cholemia, the method comprising obtaining a blood sample from a subject; adding a salt to the blood sample to form a solution; centrifuging the solution; separating the solution into a pellet and a supernatant; and comparing a color of the supernatant to a reference color of a supernatant from a normal blood sample in a reference solution with the salt to determine whether the subject has cholemia, wherein the color of the supernatant being less red than the reference color indicates that the subject has cholemia.

In certain embodiments, the subject is a human.

In certain embodiments, the method further comprises incubating the solution for a period of time before centrifuging the solution. In particular embodiments, the period of time is from about 15 minutes to about 30 minutes.

In certain embodiments, the salt comprises NaCl. In particular embodiments, the salt is present at a concentration ranging from about 0.30% by weight to about 0.48% by weight.

In certain embodiments, the salt comprises KCl. In particular embodiments, the salt is present at a concentration ranging from abou 0.55% by weight to about 0.65% by weight. In particular embodiments, the salt is present at a concentration ranging from about 0.45% by weight to about 0.5% by weight.

Further provided is a method for diagnosing cholemia, the method comprising obtaining a blood sample from a subject; adding a salt to the blood sample to form a solution; and comparing an amount of hemolysis that takes place in the solution to a reference amount of hemolysis in a normal blood sample in a reference solution with the salt to determine whether the subject has cholemia.

In certain embodiments, the determining is conducted through comparing an opacity of the solution to a reference opacity from a normal blood sample in a reference solution with the salt. In certain embodiments, the determining is conducted through comparing a color of a supernatant from the solution to a reference color of a supernatant from a normal blood sample in a reference solution with the salt. In certain embodiments, the determining is conducted through comparing a pellet size of the solution to a reference pellet size of a normal blood sample solution with the salt. In certain embodiments, the method further comprises centrifuging the solution. In particular embodiments, the method further comprises incubating the solution for a period of time before centrifuging the solution.

Further provided is an assay for testing for cholemia, the assay comprising a test strip configured to receive a blood sample; a salt solution; and a reference chart depicting at least one reference color or reference opacity. In certain embodiments, the assay further comprises a centrifuge.

Further provided is an assay for testing for cholemia, the assay comprising a plurality of test vials; a plurality of salt solutions; and a reference chart depicting at least one reference color or reference opacity. In certain embodiments, the assay further comprises a centrifuge.

Further provided is a method for evaluating resistance to lysis, the method comprising observing a difference in opacity between a first blood sample and a second blood sample, and concluding from the difference in opacity that one of the first blood sample or the second blood sample is more resistant to lysis.

Further provided is a method for evaluating resistance to lysis, the method comprising observing a difference in color between a first supernatant from a first blood sample and a second supernatant from a second blood sample, and concluding from the difference in color that one of the first blood sample or the second blood sample is more resistant to lysis.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1F: RBCs from H-TBA display resistance to osmotic hemolysis. Uncoagulated RBCs from 8-week-old male L-TBA and H-TBA mice (n=6) were collected and serum separated via centrifugation for analysis of serum total bile acids (FIG. 1A). RBCs were subjected to increasing osmotic pressure (from 0.90 to 0.00% NaCl) (FIG. 1B). Bar graphs represent osmotic fragility test of L-TBA and H-TBA RBCs in 0.50% NaCl (FIG. 1C) and 0.48% NaCl (FIG. 1D). Mice were grouped by TBA status and RBCs analyzed for osmotic fragility (FIG. 1E). FIG. 1F shows the supernatant appearance of low and high TBA RBCs incubated in 0.50% NaCl. Data represented as mean±SEM. P-values were calculated by means of Student's t-test. ****p<0.0001.

FIGS. 2A-2C: Adjustment for hematocrit does not affect hemolytic resistance in H-TBA RBCs. Uncoagulated RBCs from 8-week-old male L-TBA and H-TBA mice (n=3-6) were collected and normalized for hematocrit prior to being subjected to osmotic pressure at 0.50% NaCl (FIG. 2A), 0.42% NaCl (FIG. 2B), and 0.40% NaCl (FIG. 2C). Data represented as mean±SEM. P-values were calculated by means of Student's t-test. *p<0.05, ***p<0.001, ****p<0.0001.

FIGS. 3A-3H: Bile acid supplementation confers resistance to lysis. Uncoagulated RBCs from 8-week-old male L-TBA and H-TBA mice (n=6) were collected and plasma separated via centrifugation and switched between L-TBA and H-TBA RBCs and incubated for 1 hr at 37° C. prior to analysis of osmotic fragility (FIG. 3A) with a bar graph representing osmotic fragility of all groups at 0.50% NaCl (FIG. 1B). Uncoagulated RBCs from 8-week-old L-TBA mice (n=3) were collected and plasma separated and discarded via centrifugation. RBCs were then analyzed for osmotic fragility after 1 hr incubation at 37° C. in either cholic acid (FIGS. 3C-3E) or taurocholic acid (FIGS. 3F-3H). Data represented as mean±SEM. P-values were calculated by means of Student's t-test. *p<0.05, ***p<0.001, ****p<0.0001.

FIGS. 4A-4E: CCl₄-administered WT mice display cholemia and protection against hemolytic osmotic lysis. 8-week-old L-TBA mice were administered either carbon tetrachloride (CCl₄) (n=5) or vehicle (n=4) three times weekly via i.p. injection for 2 weeks. Uncoagulated RBCs from 8-week-old male L-TBA mice were collected two days after CCl₄ challenge and plasma separated via centrifugation for analysis of plasma TBA (FIG. 4A) and RBCs analyzed for osmotic fragility (FIGS. 4B-4E). Data represented as mean±SEM. P-values were calculated by means of Student's t-test. *p<0.05, **p<0.01, ***p<0.001.

FIGS. 5A-5F: RBC membranes have altered phospholipid content in H-TBA mice. Serum was collected from 8-week-old male L-TBA and H-TBA mice (n=4) for measurement of serum total cholesterol (FIG. 5A). Uncoagulated RBCs from 8-week-old male L-TBA and H-TBA mice (n=6) were collected and prepared as RBC ghosts to be analyzed for membrane cholesterol (FIG. 5B), phospholipids (FIG. 5C), cholesterol-to-phospholipid ratio (FIG. 5D), saturated fatty acids (SFAs) (FIG. 5E), monounsaturated fatty acids (MUFAs) (FIG. 5F), and polyunsaturated fatty acids (PUFAs) (FIG. 5G). Data represented as mean±SEM. P-values were calculated by means of Student's t-test. **p<0.01, ***p<0.001, ****p<0.0001.

FIGS. 6A-6G: RBCs from cholestatic patients are resistant to osmotic hemolysis. Uncoagulated whole blood samples were obtained from a cohort of cholestatic liver disease patients and respective non-cholestatic controls from the University of Toledo Medical Center (23 cholestatic cases and 23 non-cholestatic cases). Plasma was isolated cholestatic and non-cholestatic samples (n=10) for analysis of total bile acids (TBA) (FIG. 6A). Uncoagulated RBCs were analyzed for osmotic fragility (FIGS. 6B-6D). FIGS. 6E-6F show correlation of plasma TBA and % Hemolysis (Pearson correlation coefficient r=−0.55; p-value=0.01). FIG. 6G shows supernatant appearance of non-cholestatic and cholestatic RBCs incubated in 0.35% NaCl. Data represented as mean±SEM. P-values were calculated by means of Student's t-test. *p<0.05, ***p<0.001, ****p<0.0001.

FIGS. 7A-7C: RBCs from H-TBA mice and human cholestatic patients are protected against KCl-induced lysis. Determination of osmotic fragility for uncoagulated RBCs collected from L-TBA and H-TBA mice incubated in NaCl or NH4Cl (FIG. 7A) or KCl (FIG. 7B). FIG. 7C shows human RBCs from non-cholestatic and cholestatic patients incubated in KCl and measured for osmotic fragility. Assays were performed in replicates.

FIG. 8: Photographs of serum samples collected from human subjects. The photograph on the left shows the samples before incubation and centrifugation. The photograph in the center shows the samples after 30 minutes incubation and centrifugation. The photograph on the right shows the cell-free supernatants from the samples.

FIG. 9: Illustration depicting the osmotic lysis of red blood cells as a function of salt concentration, with sodium chloride as the salt.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

In accordance with the present disclosure, cholemic patients can be identified through a simple test of their blood. As described in the examples herein, it has been observed in both animal and human studies that cholemic blood is severely resistant to osmotic lysis. The red blood cells of subjects who are cholemic are highly resistant to hemolysis. Thus, provided herein are assays and methods for detecting or diagnosing cholemia in a subject, which utilize the correlation between cholemia and resistance to osmotic lysis.

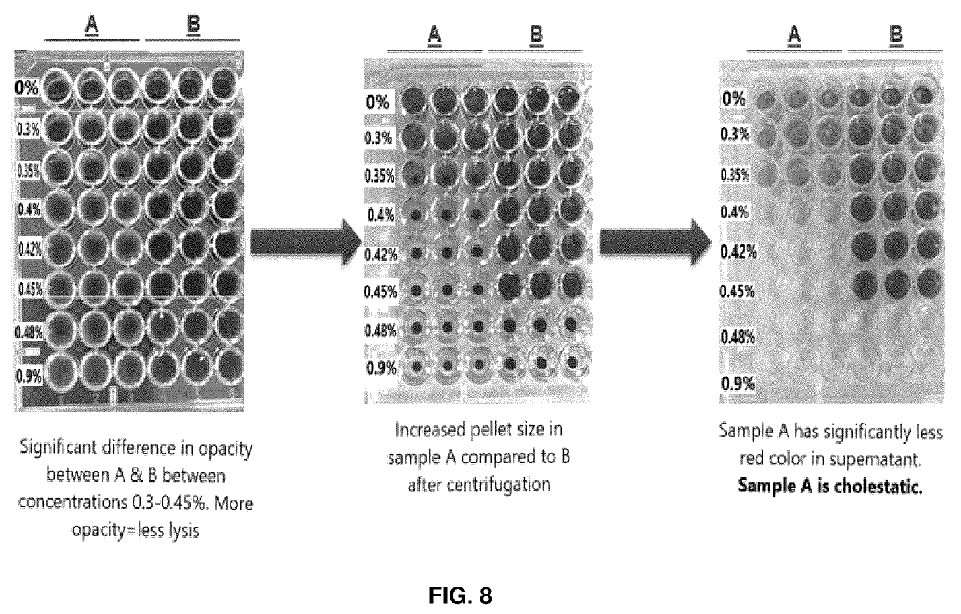

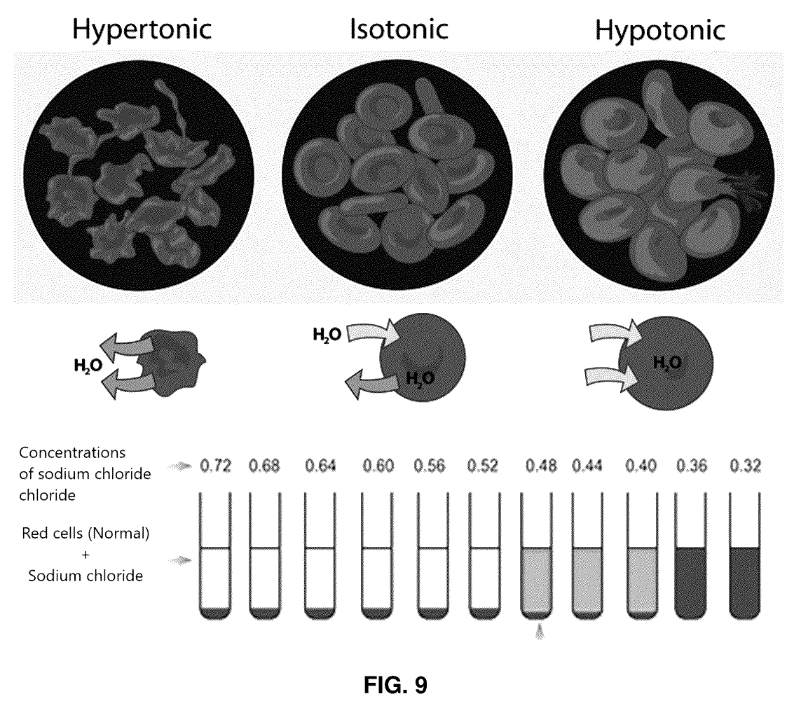

A sample of blood may be obtained from a subject, such as a human or animal subject. A salt may be added to the sample of blood, and the resulting solution may be allowed to incubate for a period of time such as, but not limited to, about 15 minutes, or about 30 minutes. However, the incubation is not strictly necessary. Upon exposure of red blood cells in a blood sample to a salt, osmotic hemolysis generally occurs due to an osmotic imbalance that causes excess water to diffuse into the cells. During this process, red blood cells rupture and release their contents into the surrounding fluid. However, cholemic blood is resistant to osmotic hemolysis, and therefore will produce a lesser degree of osmotic hemolysis than normal blood upon exposure to the same salt conditions. As illustrated in FIG. 8, this enables the use of a simple comparison, using either opacity or color, between the blood sample and a reference sample to diagnose cholemia.

In a first aspect, a difference in opacity can be utilized to diagnose a subject as having cholemia from a blood sample, as seen in the image on the left in FIG. 8. The opacity of the blood sample in solution with the salt can be analyzed without further processing of the sample. The practitioner need only observe the opacity of the blood sample in solution with the salt and compare it to the opacity of a normal blood sample in a salt solution, which may be represented pictorally (such as in a reference chart showing a reference opacity) to avoid the need for analyzing a second physical sample. A greater amount of opacity in the sample compared to the reference sample indicates that the sample has undergone less osmotic hemolysis, and therefore contains excess bile acids. The more lysis that has occurred in a blood sample, the more light that will pass through the blood sample. Thus, blood cells which are highly resistant to hemolysis will be more opaque than blood cells which are not highly resistant to hemolysis. Accordingly, a greater amount of opacity in the sample compared to the reference sample indicates that the subject has cholemia.

In a second aspect, a difference in the color of the supernatant following centrifugation can be utilized to diagnose cholemia in the blood sample, as seen in the image on the right in FIG. 8. The blood sample obtained from the subject can be incubated in the salt solution for a period of time such as about 15 minutes or about 30 minutes (though this is not strictly necessary), and then centrifuged and separated into a pellet and a supernatant. The color of the supernatant can then be compared (either visually or with an instrument such as a spectrophotometer) to a reference color from a normal blood sample supernatant having been exposed to the same salt solution and centrifuged. The reference color may be provided pictorally such as in a reference chart. When the observed color of the sample supernatant is less red than the reference color, it indicates that the sample has undergone less osmotic hemolysis, and therefore contains excess bile acids. Accordingly, a less red color of the sample compared to the reference sample indicates that the subject has cholemia.

Though opacity and color are merely two example ways of determining the extent to which osmotic hemolysis has occurred in the blood sample, so as to determine whether a sample is resistant to osmotic hemolysis, it is understood that other ways of determining the extent to which osmotic hemolysis has occurred in the blood sample are possible and encompassed within the scope of the present disclosure. As one non-limiting example of an alternative, as seen in the image in the center in FIG. 8, the pellet size following centrifugation may be indicative of cholemia, where the pellet size is increased in cholemic blood upon incubation with salt and centrifugation compared normal blood.

Regardless of how the extent of osmotic hemolysis is determined, the salt used to provoke osmotic hemolysis in the blood sample can include any salt or other lysis-inducing agents which rely on osmosis, and can be in the form of a solution added to the blood sample. Non-limiting examples of salt solutions include saline (i.e., NaCl in water), and KCl solutions. The salt solutions may optionally include an anticoagulant such as heparin or EDTA. Many other salts are possible. Notably, however, salts such as ammonium chloride, which cause lysis but through mechanisms other than osmosis, are not ideal for use in the methods and assays described herein.

It is understood that the concentration of the salt may depend on the composition of the salt, and is not particularly limited so long as the concentration of salt used causes osmotic hemolysis in normal blood. For example, when the salt comprises NaCl, the salt may be added at a concentration ranging from about 0.30% by weight to about 0.48% by weight, or from about 0.45% by weight to about 0.5% by weight. As another example, when the salt comprises KCl, the salt may be added at a concentration ranging from about 0.55% by weight to about 0.65% by weight, or from about 0.45% by weight to about 0.5% by weight.

Various assays may be employed to practice the methods described herein. As a non-limiting example, an assay may include a test strip configured to receive a blood sample, a salt solution, and a reference chart showing one or more reference opacities or colors. As another non-limiting example, an assay may include a plurality of test vials and a plurality of salt solutions to facilitate testing and comparing an unknown sample and a control sample simultaneously, along with a reference chart showing one or more reference opacities or colors. In some embodiments, the assays may include a centrifuge. In some embodiments, the assays may include an apparatus or instrument for extracting a blood sample from a subject.

Advantageously, the assays and methods described herein are accurate both in asymptomatic and symptomatic patients, and are cheap, simple, and non-invasive.

### Examples

### Systemically Elevated Bile Acids Induce Osmotic Lysis Resistance in Erythrocytes

In these examples, the consequences of cholemia and cholestasis on erythrocyte cellular physiology were evaluated. A murine model of idiopathic cholemia was employed, and samples collected from cholestatic patients were utilized. The cholemic mouse model has naturally occurring cholema, and no abnormal liver function or presence of bilirubin, but has elevated quantities of bile acids. Human subjects were then analyzed as well. In contrast to previous findings, cholemic mice and cholestatic patients with high circulating total bile acids (≥40 M) were strikingly resistant to ionic salt-induced hemolysis when compared to their healthy counterparts. A significant inverse relationship was observed, where healthy mice with normal physiological bile acid levels had the strongest hemolysis reaction compared to the greatest protection seen in mice with toxic bile acid levels. Introducing bile acids to healthy RBCs either by plasma from cholemic mice or exogenous supplementation of unconjugated cholic acid gave the susceptible RBCs more resistance to hemolysis. Interestingly, cholemic mice had intact cholesterol levels, depleted phospholipids, and a shift in the fatty acid composition that favored monounsaturated rather than saturated fatty acids, which may be collectively promoting membrane fluidity. Overall, this redefines how bile acids impact RBC membrane integrity. The results show that cholemic blood is highly resistant to osmotic hemolysis, and this can be used to diagnose or screen for cholemia through simple comparisons of blood samples.

### Materials and Methods

### Mice

C57BL/6J wild-type (WT) mice (Stock #000664) were procured from Jackson Laboratory and bred in-house in specific pathogen-free condition at the University of Toledo College of Medicine and Life Sciences. Mice were housed in cages (n=5 mice/cage) containing corn cob bedding (Bed-O-Cob; The Andersons Co.) and nestlets (Cat #CABFM00088; Ancare). The cages were housed at 23° C. under a 12/12-h light/dark phase cycle and fed a grain-based laboratory chow diet (LabDiet 5001) ad libitum. Mice were screened for serum total bile acids (TBA) prior to weaning. On day 22 for weaning, mice with low bile acids (L-TBA<40 M) or high bile acids (H-TBA≥40 M) were segregated into separate cages. To induce liver fibrosis in mice, carbon tetrachloride (Sigma-Aldrich, St. Louis, MO) (0.5 L/g body weight) was administered intraperitoncally three times a week for 2 weeks with corn oil (Sigma-Aldrich) (50 μL) serving as vehicle control.

### Measures of Hematologic Parameters

Blood was collected through submandibular bleeding. EDTA-containing Vacuette (Greiner bio-one) tubes were used to store the blood for the immediate measurement of complete blood count (CBC) by VETSCAN HM5 Hematology Analyzer (ABAXIS), which includes a 22-parameter CBC result panel including the measurements for RBC-related parameters such as RBC counts, Hgb, HCT, MCV, MCH, and MCHC.

### Serum Collection

Blood from 8-wk-old male mice was collected into BD microtainers (BD Biosciences). Samples were centrifuged at 10,000 rpm for 10 min at 4° C. and hemolysis-free sera was collected. Serum was stored at −80° C. until further analysis.

### Serum Total Bile Acid Quantification

Total bile acids (TBA) in hemolysis-free sera were measured using a TBA assay kit (Enzyme Cycling Method; Diazyme Laboratories) according to the manufacturer's protocol.

### Osmotic Fragility Test

Blood was collected in EDTA-containing tubes and then 10 μl aliquots were placed into a round bottom 96-well plate. The number of aliquots correlated to the number of concentrations per buffered NaCl (Fisher Scientific, Hampton, NH), KCl (Sigma-Aldrich), and NH₄Cl (Sigma-Aldrich) salt solutions, respectively |NaCl (%) 0, 0.30, 0.35, 0.40, 0.45, 0.48, 0.50, and 0.90; KCl (%) 0, 0.40, 0.42, 0.45, 0.48, 0.50, 0.55, 0.60-0.65, 0.70, and 0.90; NH₄Cl (%) 0, 0.40, 0.42, 0.45, 0.48, 0.50, and 0.90]. The respective concentration and type of salt solution was added to the samples (300 μl). The plate was gently mixed and incubated for 30 min at room temperature. Samples were centrifuged at 1,300×g for 10 min and supernatant was transferred into a 96-well flat bottom plate. The degree of red blood cell (RBC) hemolysis was evaluated spectrophotometrically at 540 nm on a Biotek Eon microplate spectrophotometer and normalized to the optical density value of 0.0% buffered salt solution. For investigating the effects of plasma switch on RBCs, uncoagulated blood was collected from L-TBA and H-TBA mice and centrifuged for isolation of plasma. L-TBA RBCs were incubated in H-TBA plasma and H-TBA RBCs incubated in L-TBA plasma at 37° C. for 1 hr prior to measurement of osmotic fragility. For ex-vivo bile acid supplementation in RBCs, uncoagulated blood was collected from L-TBA mice, plasma removed and discarded, and remaining RBCs gently resuspended in either 100 μM of cholic acid sodium salt (Sigma-Aldrich) or taurocholic acid sodium salt hydrate (Alfa Aesar, Ward Will, MA) dissolved in phosphate-buffered saline (PBS), or PBS alone. All groups were incubated at 37° C. for 1 hr prior and measured for osmotic fragility.

### Preparation of Red Blood Cell Ghosts

Red blood cell (RBC) membranes (ghost) were prepared as follows. Briefly, after removal of the plasma, isolated RBCs were suspended and mixed gently in isotonic Tris-HCl buffer (0.172 M, pH 7.6, 4° C.). The cell suspension was centrifuged at 1,000×g for 10 min at 4° C. and the supernatant was discarded. RBCs were hemolyzed in hypotonic Tris HCl buffer (0.011 M, pH 7.6, 4° C.) and allowed to stand for 5 min on ice before centrifugation at 20,000×g for 20 min at 4° C. The supernatant was carefully removed without disrupting the RBC membranes (pellet). Membranes were repeatedly washed with 0.011 M Tris-HCl buffer until their color turned to creamy white. On the last wash, the supernatant was removed and RBC ghosts were stored at −80° C. until analysis.

### Analysis of RBC Ghost Phospholipids and Cholesterol

Membrane lipid analysis on RBC ghosts was performed at mouse metabolic phenotyping centers (MMPC) Lipid Core Laboratory, Vanderbilt University. Lipid concentration was normalized to total protein of the RBC ghosts.

### Human Study

Human cholestatic liver disease and control (without any liver disease) blood samples were collected and provided by The Department of Pathology at The University of Toledo. Blood samples were collected either in EDTA or heparin containing tubes. Cholestatic patients were selected on the basis of meeting at least one or more parameters associated with cholestasis: bilirubin>2.5 mg/dL; gamma-glutamyltransferase (GGT)>30 U/L; alanine aminotransferase (ALT)>50 U/L; aspartate aminotransferase (AST)>36 U/L; lactate dehydrogenase (LDH)>225 U/L. There were a total of 46 participants for the study (23 samples were designated as cholestatic and 23 samples were non-cholestatic controls). Male patients represented 65.2% (median age: 60 years) while female patients represented 34.8% (Median age: 59 years) of the entire cohort.

### Statistical Analysis

The results are expressed as mean±SEM. Statistical analysis for significance between two groups was conducted using Student's t-test (unpaired, two-tailed) with *p<0.05 considered as significant. The Spearman correlation (r) was used to establish the association of two variables. All statistical analyses were performed with the GraphPad Prism 9.0 program (GraphPad Software, Inc, La Jolla, CA).

### Results

Red Blood Cells from H-TBA Mice Display Resistance to NaCl-Induced Osmotic Hemolysis

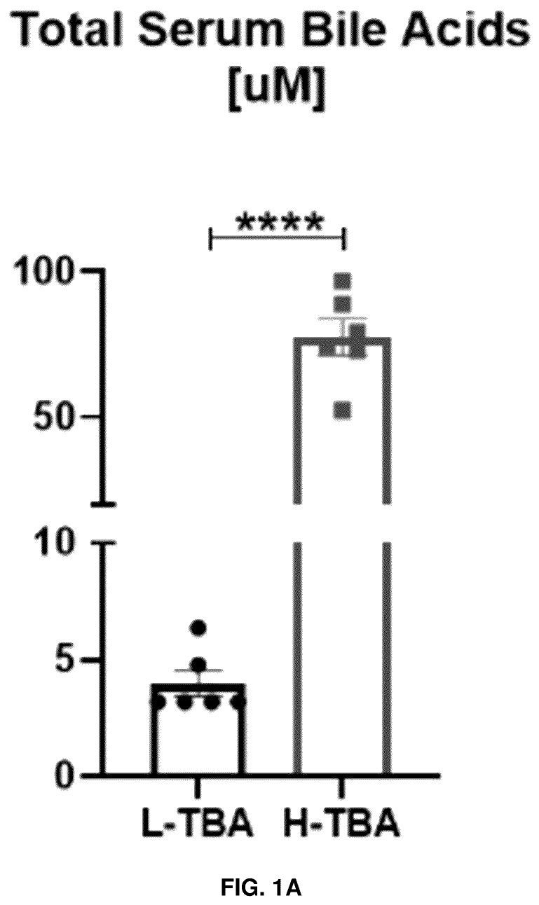

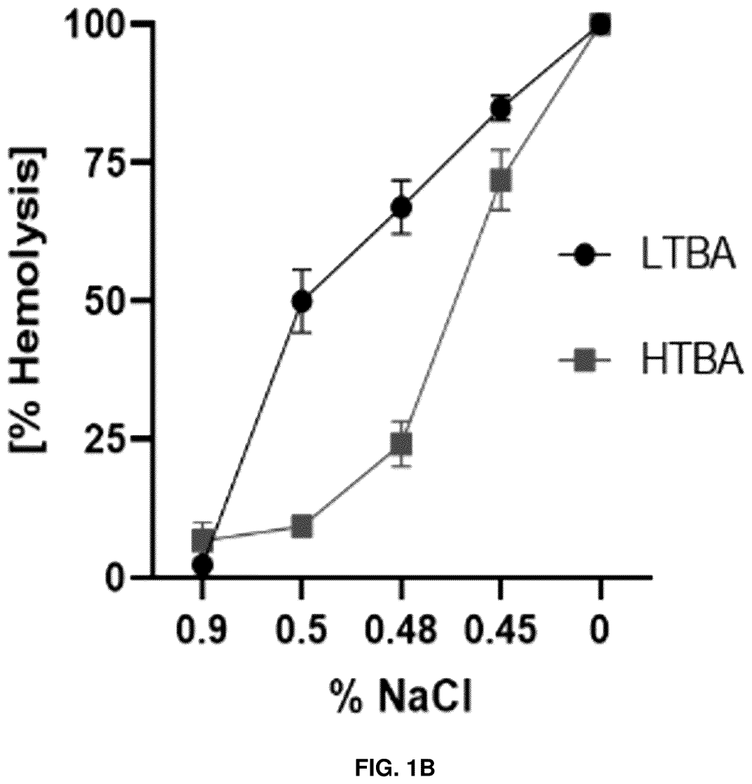

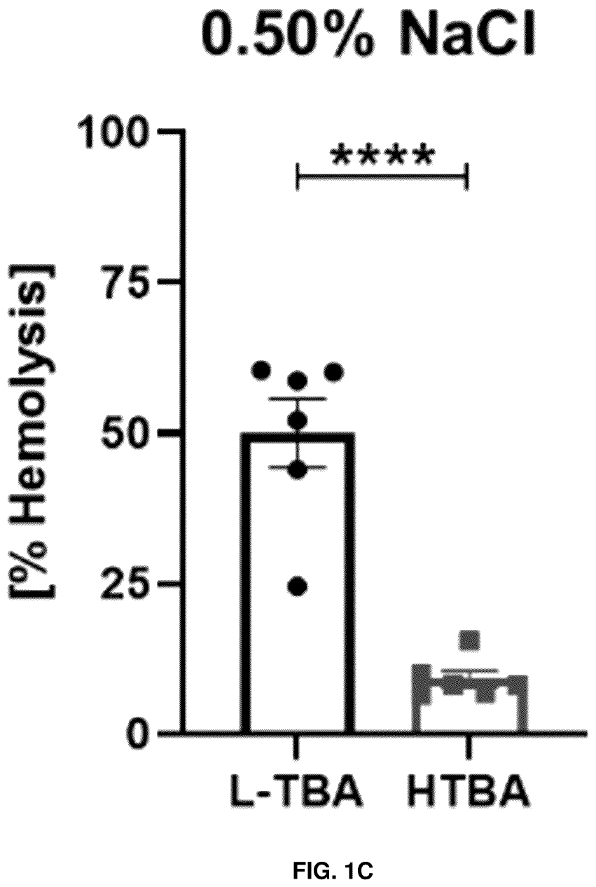

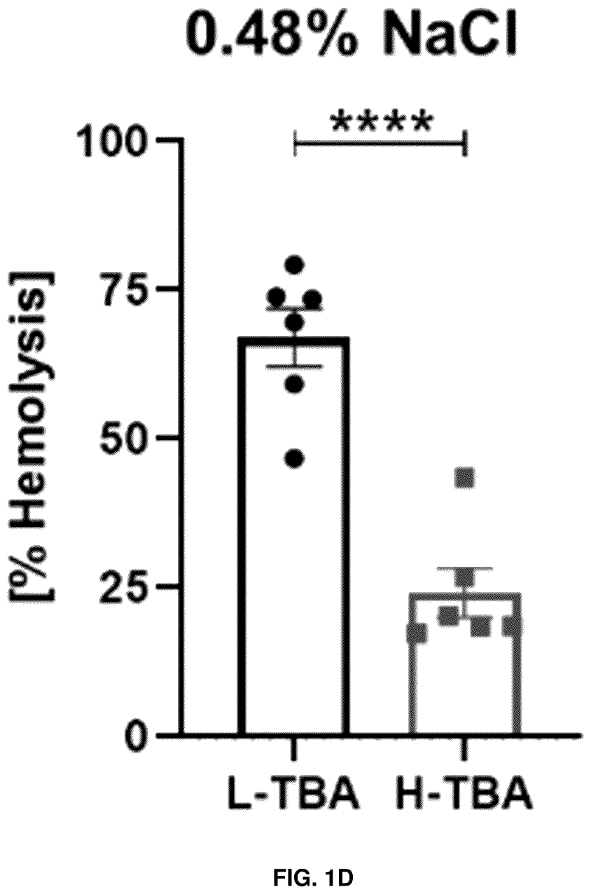

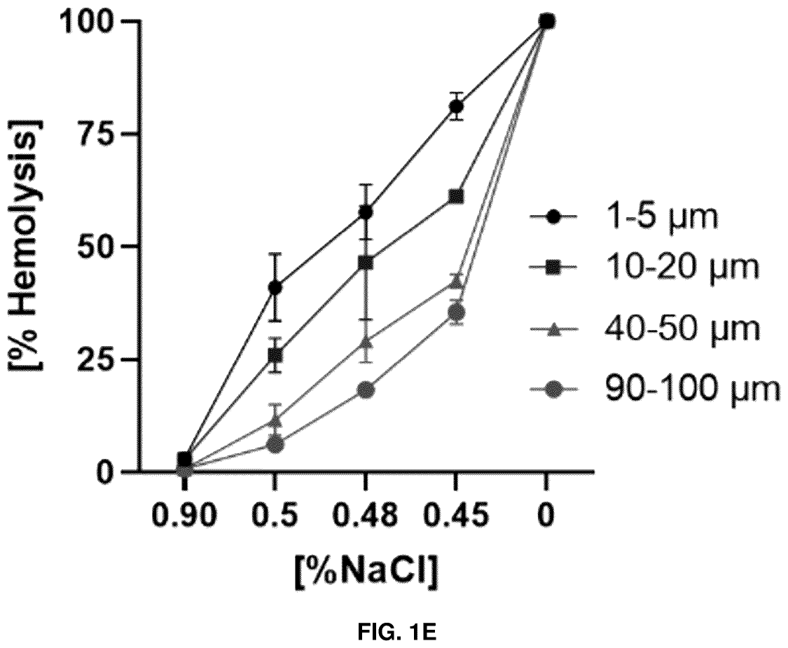

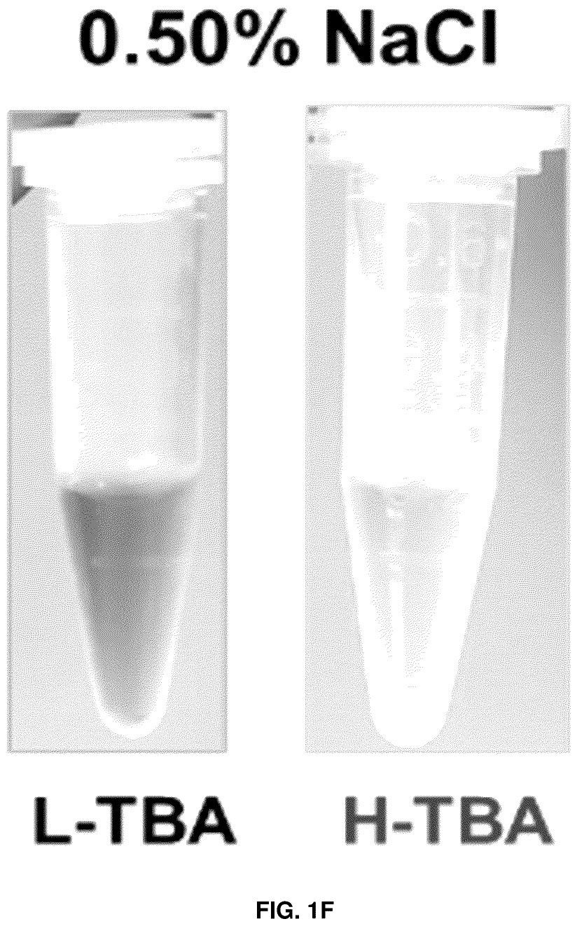

Bile acids disrupt live plasma cell membranes and synthetic cell membranes. These prior observations were determined by collecting intact cells or isolating cell membranes from a mammal (e.g., rat, pig, sheep, rabbit, human), incubating the samples with a specific concentration of an individual bile acid, and measuring the level of hemolysis. In these experiments, whether chronic exposure to high total bile acid (TBA) levels exceeding the physiological threshold would induce severe membrane damage in a mammal was tested. A rodent model of a subset (˜10%) of wild-type (WT) C57BL/6 mice that exhibit spontaneous portosystemic shunt was utilized to do this. WT mice with portosystemic shunt have elevated serum total bile acids (TBA) and are stratified with the status of high-TBA (≥40 μM) compared to low-TBA (L-TBA<40 μM) mice without portosystemic shunt (FIG. 1A). As seen in FIG. 1A, a 20-fold increase in total serum bile acids was observed.

It can be surmised that continued exposure to elevated TBA would alter every cell membrane upon contact, but red blood cells (RBC) were used as a surrogate cell type to determine cell membrane integrity in H-TBA mice. First, a complete blood count (CBC) analysis was performed to investigate for any substantial deviations in RBC parameters. Notably, H-TBA mice appeared to have mild anemia because of the significantly reduced hemoglobin (Hgb), hematocrit (HCT), mean corpuscular volume (MCV), and mean corpuscular hemoglobin (MCH) values (Table 1). As anemia can be a consequence of hemolysis, sodium chloride (NaCl)-induced osmotic stress was next employed to measure RBC integrity and fragility. Surprisingly, RBCs from H-TBA mice were remarkably resistant to hemolysis across a wide range of NaCl concentrations (FIG. 1B). Of note, the most hemolytic resistance in H-TBA mice was found at the 0.48% and 0.50% NaCl concentrations (FIGS. 1C-1D), and the lysis susceptibility versus resistance phenotype was inversely correlated with serum TBA levels (FIG. 1E). A visually impressive sharp contrast was also seen in the color of the supernatant between L-TBA and H-TBA RBCs incubated with 0.50% NaCl (FIG. 1F). Compared to the bright red color in the L-TBA supernatant, the hemolytic resistance in H-TBA mice corresponded with a very light red tint that was near transparent. Importantly, when performing osmotic lysis and looking at the supernatant color in a blinded fashion, L-TBA and H-TBA mice were able to be retrospectively identified without needing to measure TBA beforehand. Collectively, these results emphasize that high TBA levels promote RBC membrane resistance to hemolysis.

TABLE 1
H-TBA mice manifest indices of anemia

| Parameters | LTBA | HTBA |
|---|---|---|
| WBC (10⁹/L) | 7.4 ± 1.2 | 9.0 ± 2.3 |
| Neutrophils (10⁹/L) | 1.6 ± 0.3 | 1.7 ± 0.4 |
| Lymphocytes (10⁹/L) | 5.4 ± 0.9 | 6.8 ± 1.8 |
| Monocytes (10⁹/L) | 0.4 ± 0.1 | 0.6 ± 0.2 |
| RBC (10¹²/L) | 9.8 ± 0.3 | 8.9 ± 0.2* |
| Hgb (g/dL) | 13.4 ± 0.4 | 11.2 ± 0.4** |
| HCT (%) | 43.3 ± 1.1 | 36.5 ± 1.1*** |
| MCV (fl) | 44.3 ± 0.2 | 41.5 ± 0.3**** |
| PLT (10⁹/L) | 180.8 ± 51.9 | 193.8 ± 49.3 |
| MCH | 13.7 ± 0.1 | 12.6 ± 0.2*** |
| MCHC | 30.9 ± 0.3 | 30.5 ± 0.5 |

Data represented as mean ± SEM. P-values were calculated by means of Student's t-test.
*p < 0.05,
**p < 0.01,
***p < 0.001,
****p < 0.0001.

Data represented as mean±SEM. P-values were calculated by means of Student's t-test. *p<0.05, **p<0.01, ***p<0.001, ****p<0.0001.
Adjusting for Hematocrit Levels in H-TBA Mice does not Affect Resistance to Osmotic Hemolysis

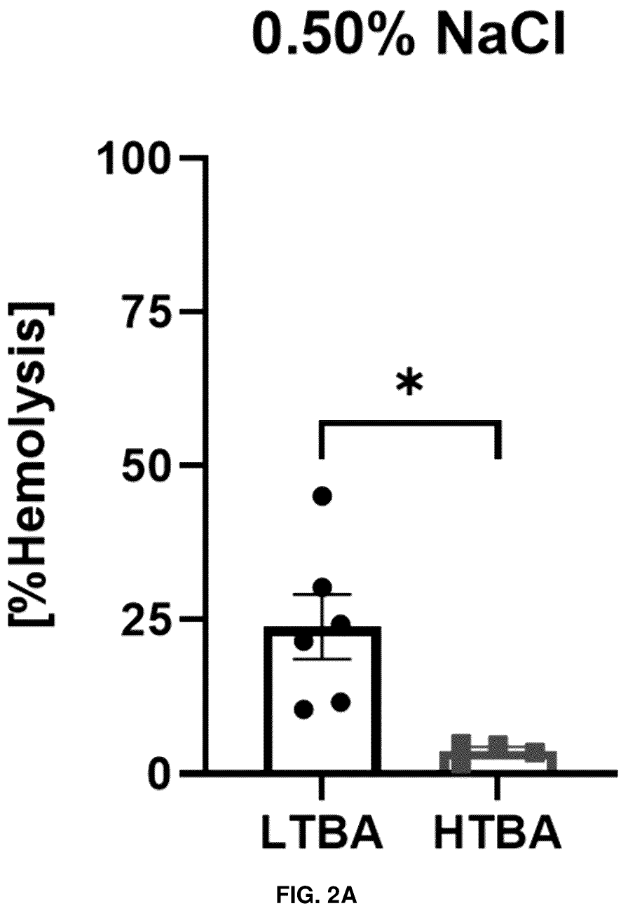

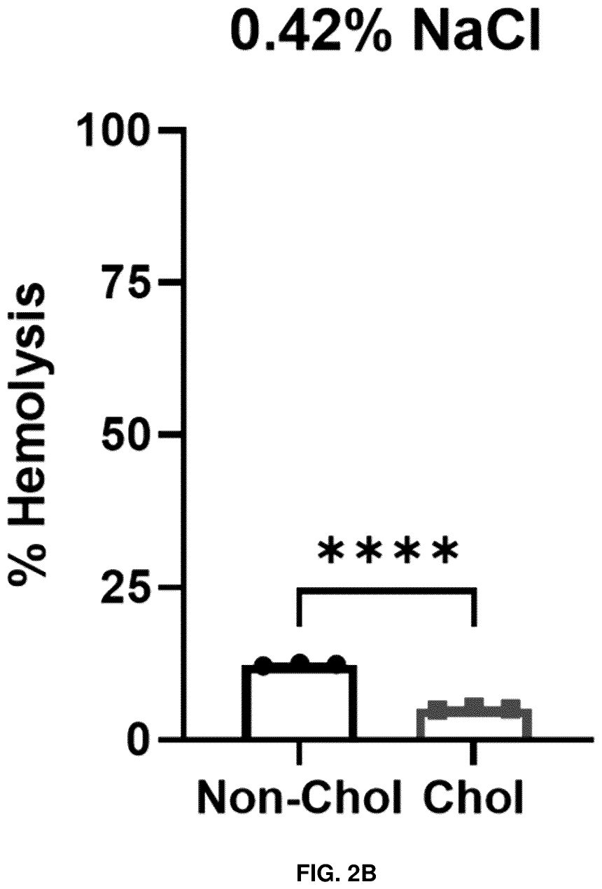

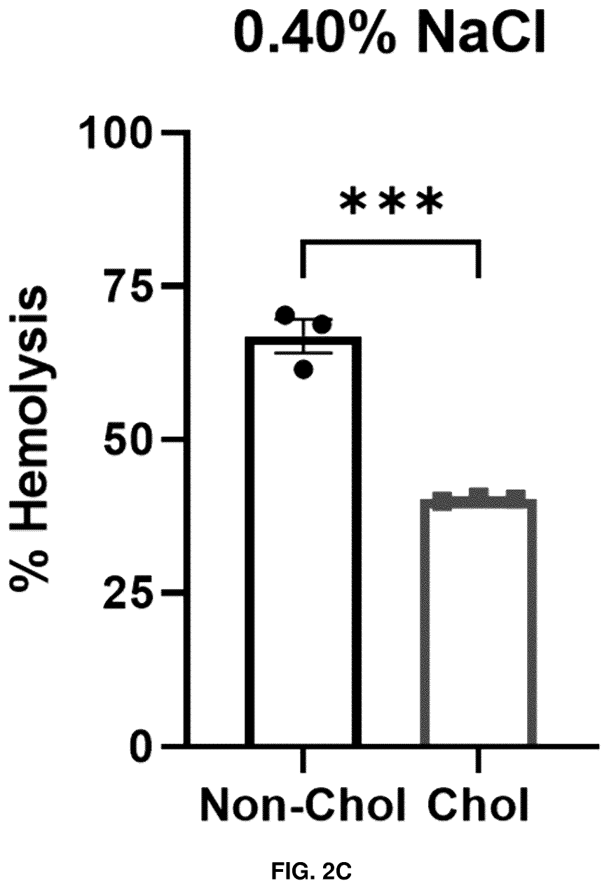

It is important to rule out whether the hemolysis resistance phenotype in H-TBA mice was attributed to a lack of RBCs because the osmotic fragility assay crudely measures absorbance based on free hemoglobin released in the supernatant. To circumvent this, the mean HCT values between L-TBA and H-TBA mice were used to normalize for hematocrit values in the assay. As Table 1 showed a 16% reduction of HCT in H-TBA mice, 16% more blood volume was added for the assay in order to adjust and measure for osmotic hemolysis. Intriguingly, H-TBA RBCs still maintained resistance to lysis even after HCT normalization (FIG. 2A), confirming the phenotype was due to prolonged bile acid exposure rather than anemia.

### Supplementation of Total or Unconjugated Bile Acids Confers Resistance to Lysis

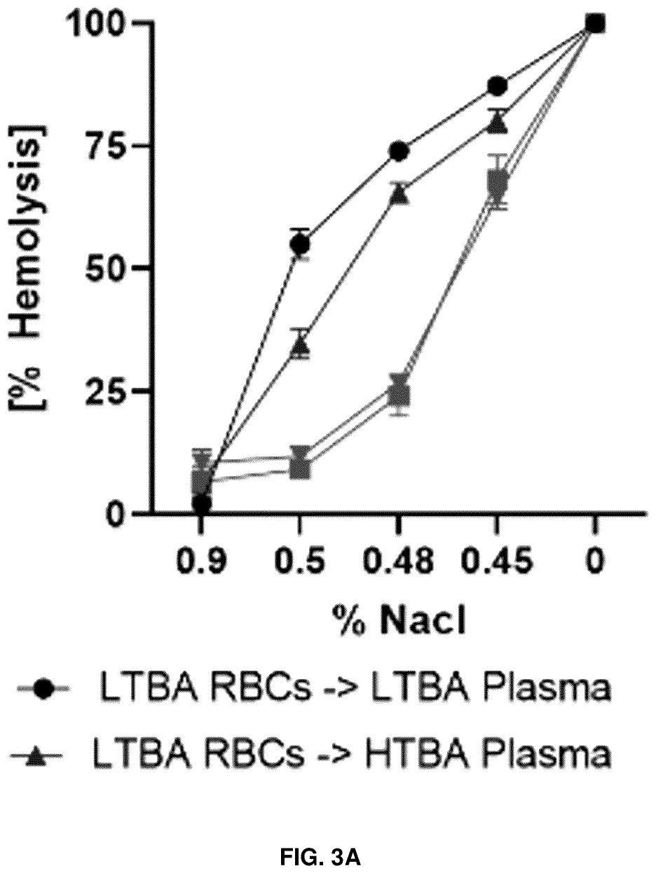

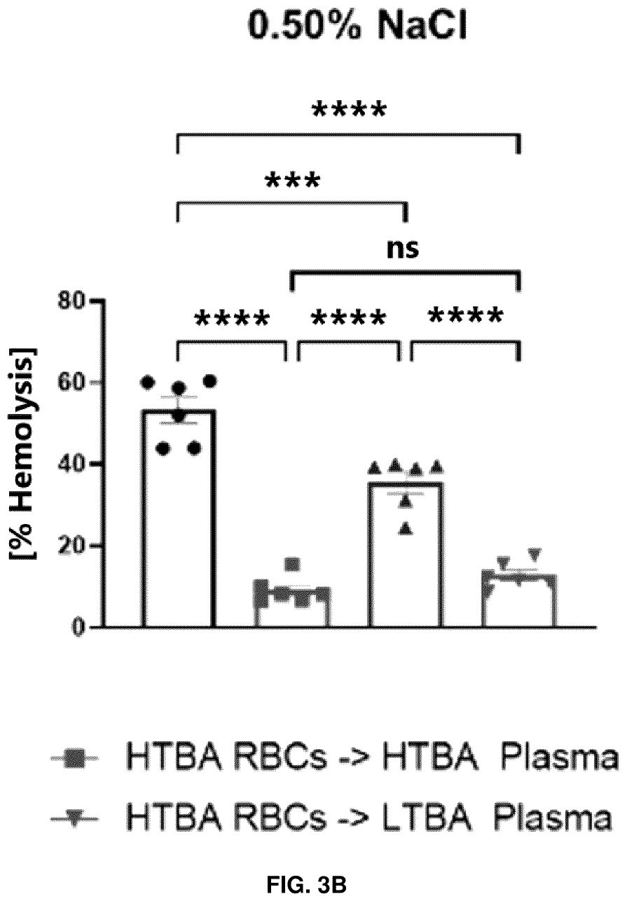

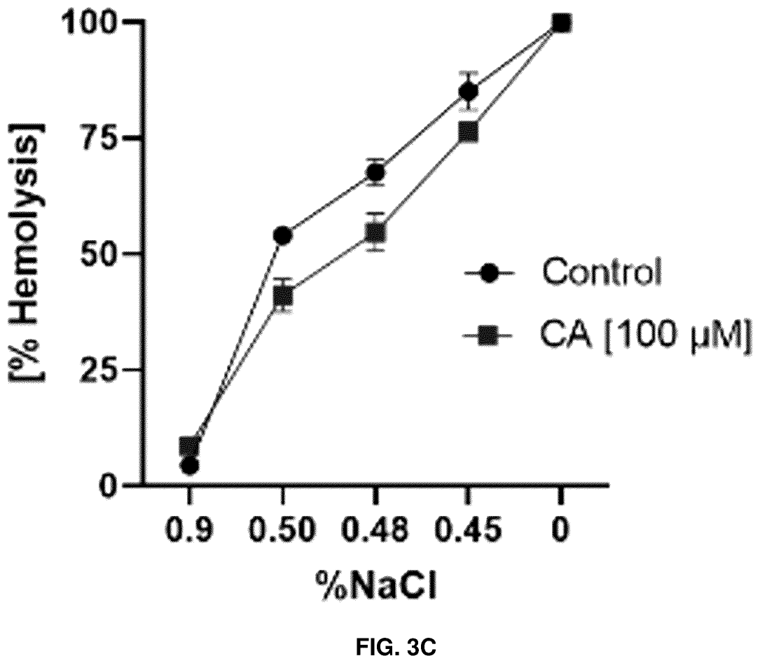

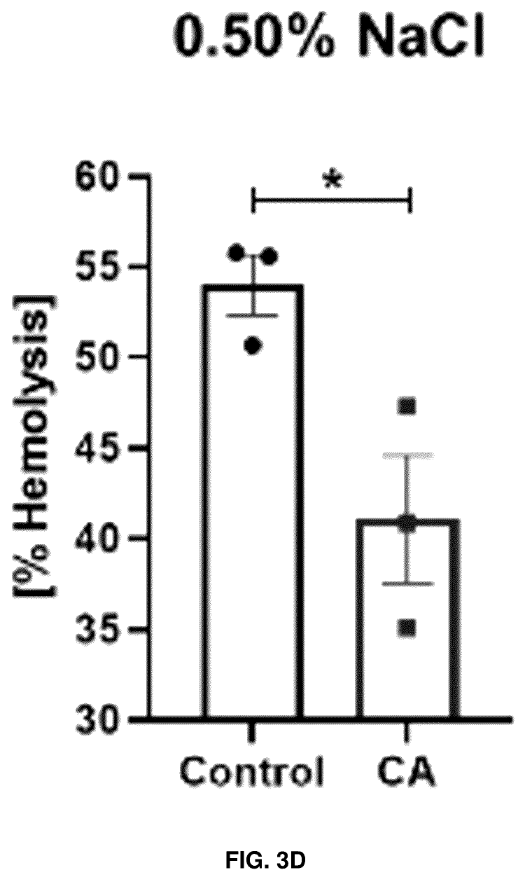

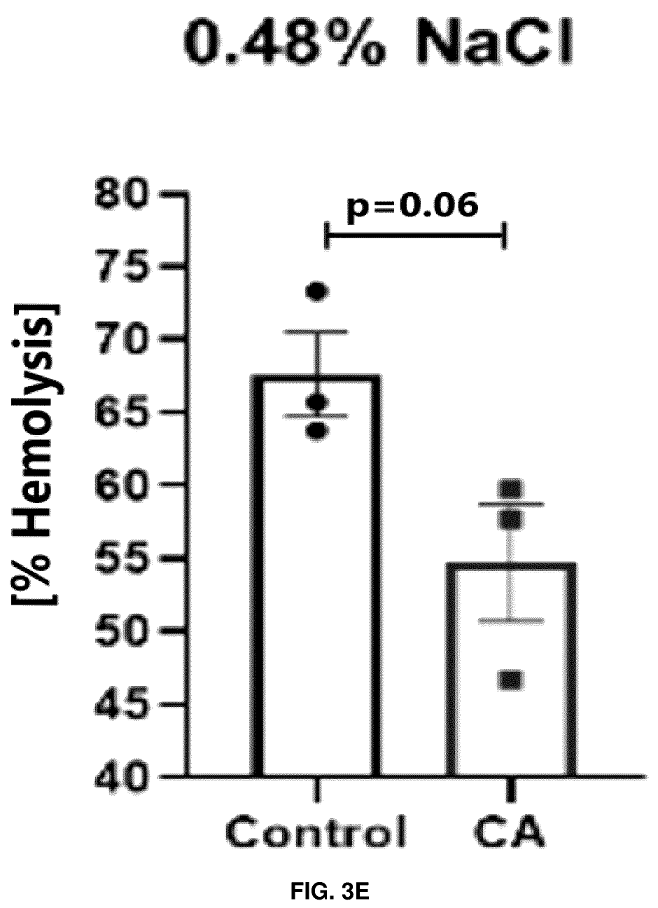

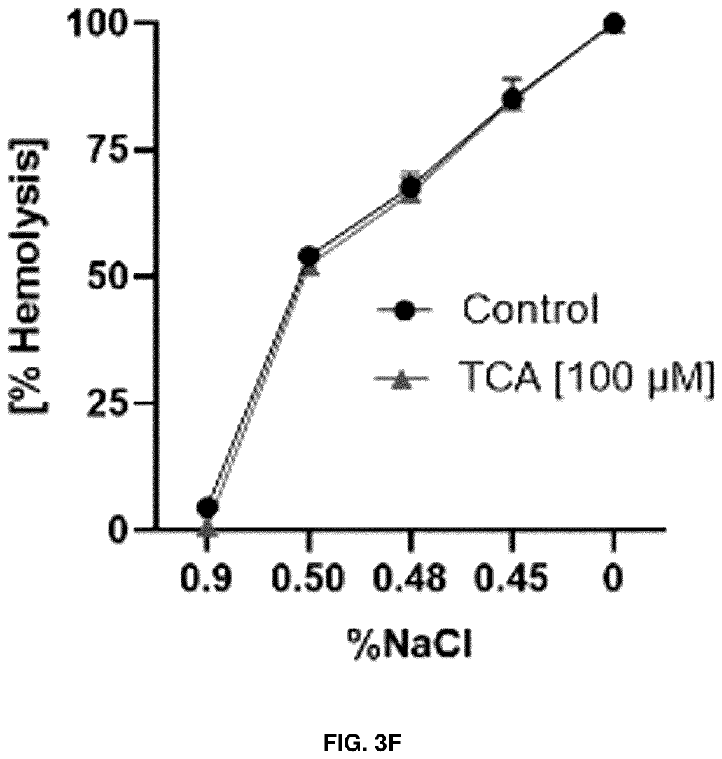

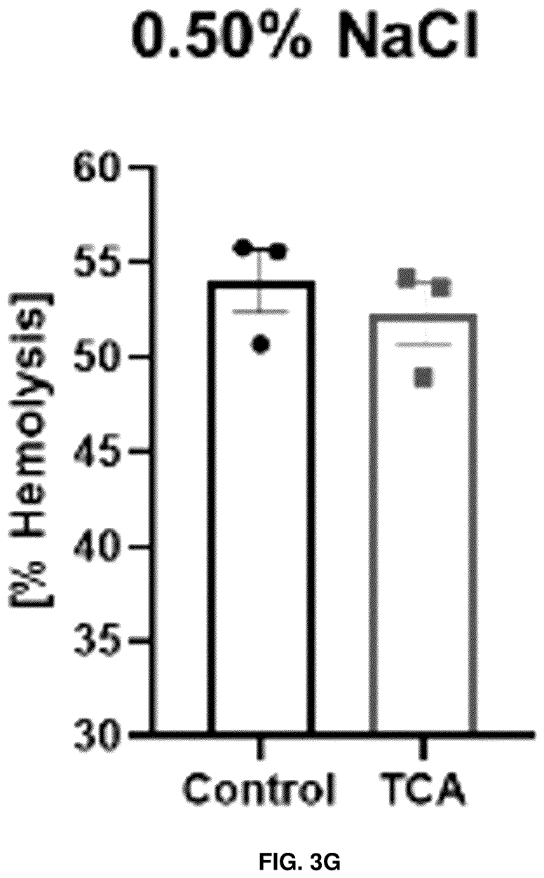

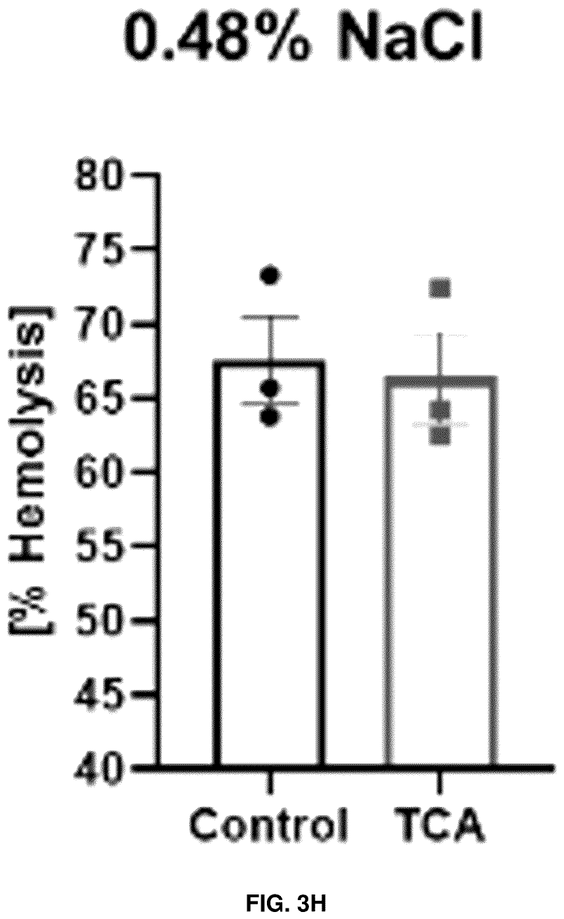

The extent to which hemolysis resistance was attributed from direct contact with bile acids was investigated. As RBCs from L-TBA mice were highly susceptible to hemolysis, bile acid-enriched plasma from H-TBA mice was introduced to the L-TBA RBCs and vice versa as a form of plasma switch. Intriguingly, H-TBA RBCs incubated in L-TBA plasma had no effect on RBC fragility, but L-TBA RBCs incubated in H-TBA plasma became more resistant to lysis (FIGS. 3A-3B). In a similar fashion, incubating L-TBA RBCs with exogenous supplementation of cholic acid (most predominant bile acid in mammals) could confer resistance to the same level as observed in the plasma switch (FIGS. 3C-3E). Importantly, this cholic acid-induced hemolytic resistance only occurred when the bile acid was introduced in its unconjugated form, whereas the conjugated taurocholate failed to induce any resistance but, rather, had the same lysis susceptibility as L-TBA RBCs (FIGS. 3F-3H). Without wishing to be bound by theory, it is believed that this is due, in part, to conjugated bile acids being hydrophilic whereas the hydrophobic property of unconjugated, protonated bile acids allows for efficient ‘flip-flop’ transbilayer movement across the erythrocyte membrane, and this may subsequently alter the RBC membrane structure to increase lysis resistance.

### Carbon Tetrachloride-Induced Liver Injury Promotes Resistance to Osmotic Lysis

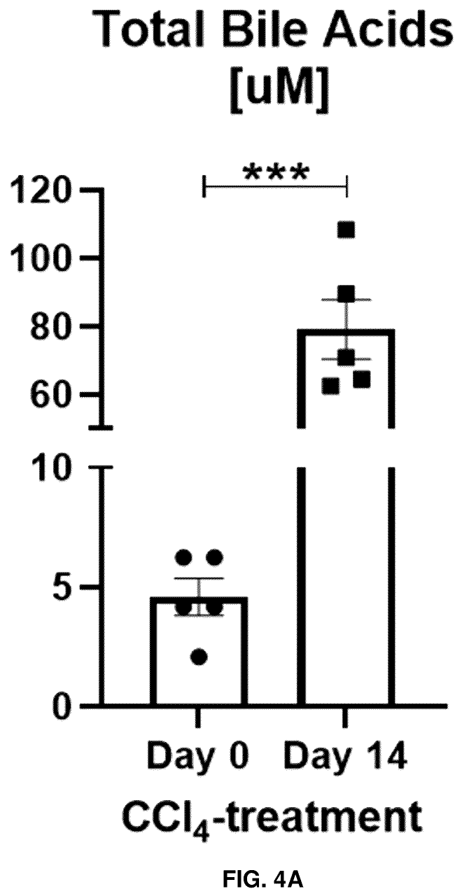

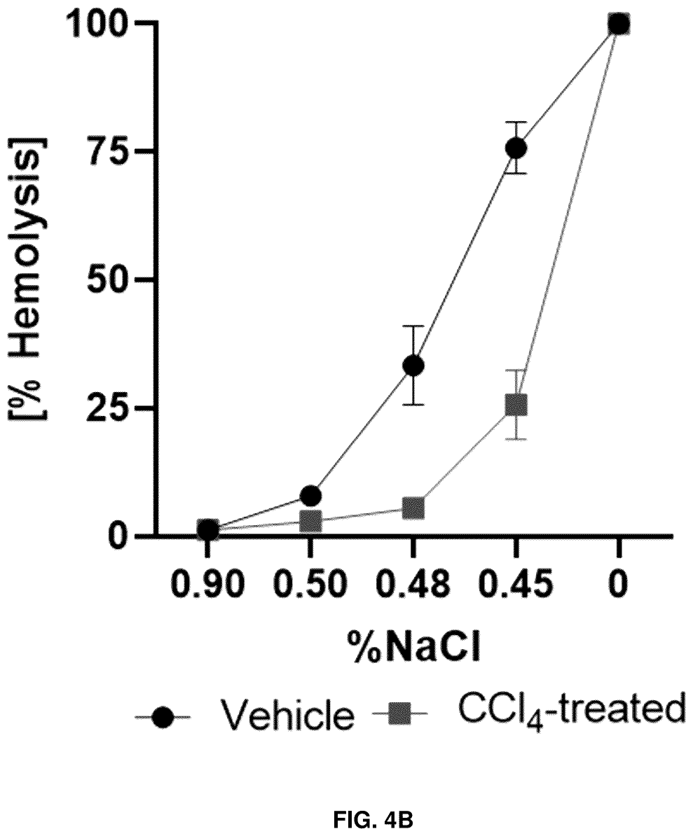

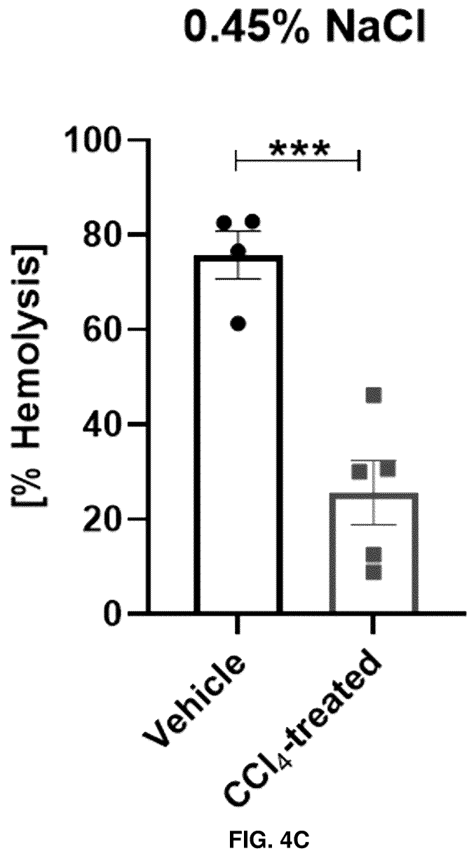

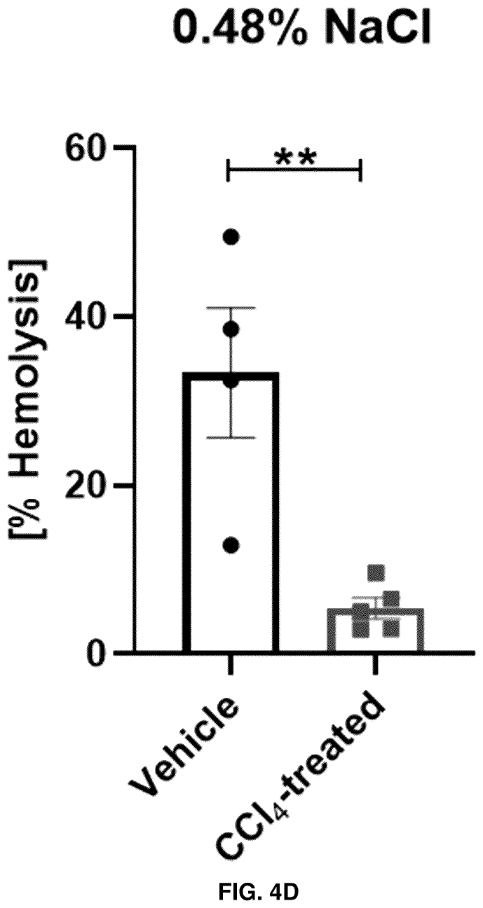

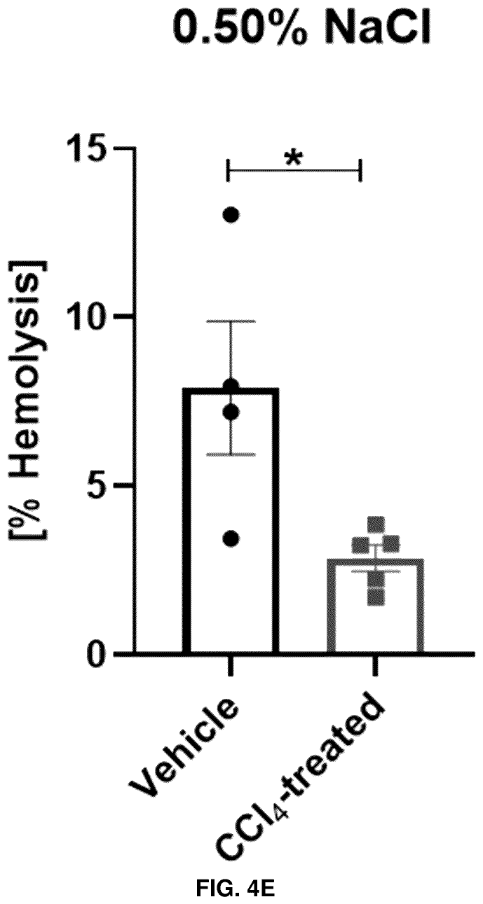

Drug-induced liver injury (DILI) is the leading cause of acute liver failure globally. Interestingly, 20-40% of all DILI case patterns are cholestatic stemming from inhibition of hepatobiliary bile transporters. Therefore, examining RBC fragility in mice with DILI is useful. The well-characterized carbon tetrachloride (CCl₄)-induced liver fibrosis model, which has been previously noted for inducing cholemia in rats, was utilized. L-TBA mice were treated with either CCl₄ or vehicle control for 2 weeks, and a 16-fold increase in serum TBA levels in CCl₄-administered mice was observed (FIG. 4A). In measuring for osmotic fragility, it was observed that RBCs CCl₄-treated mice were highly resistant to osmotic lysis (FIGS. 4B-4E), further indicating that systemically elevated BA levels promote osmotic lysis resistance in vivo.

H-TBA RBC Membranes have Altered Lipid Content

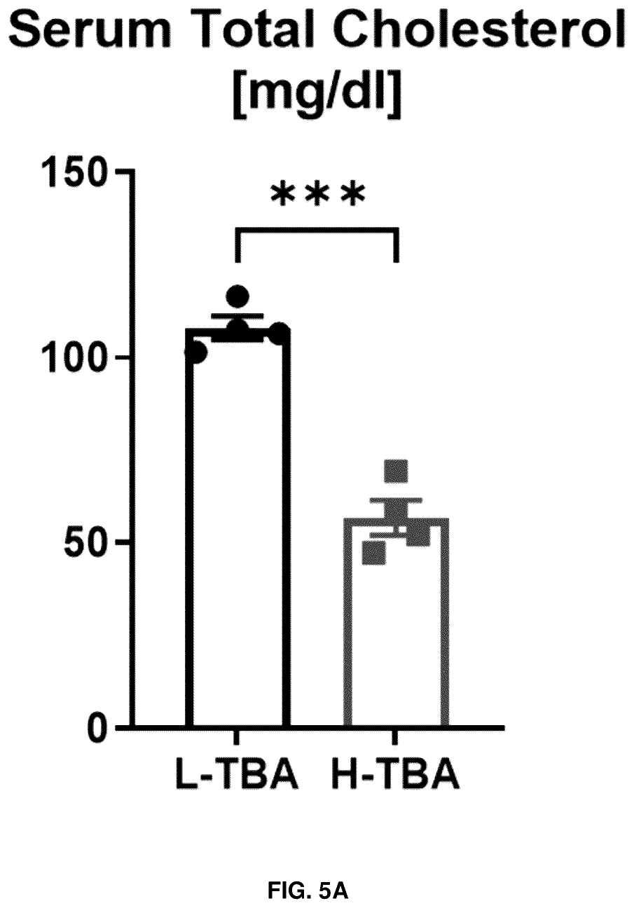

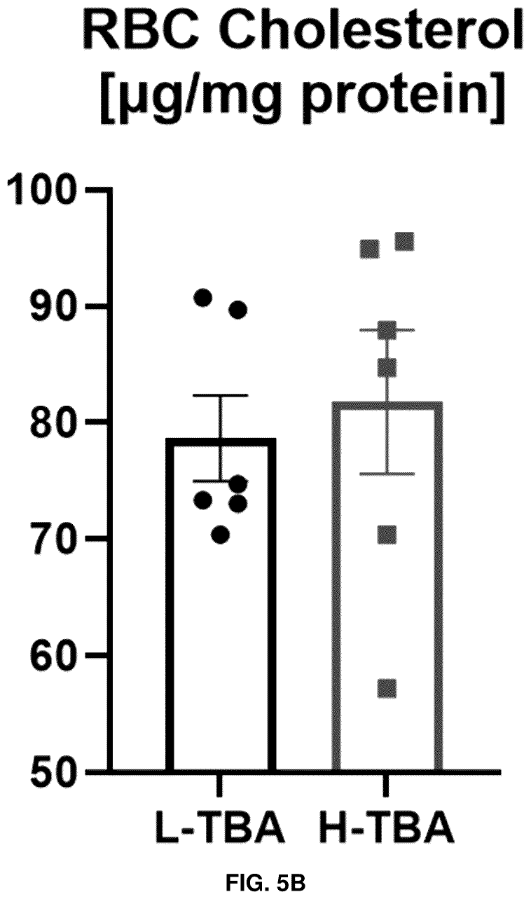

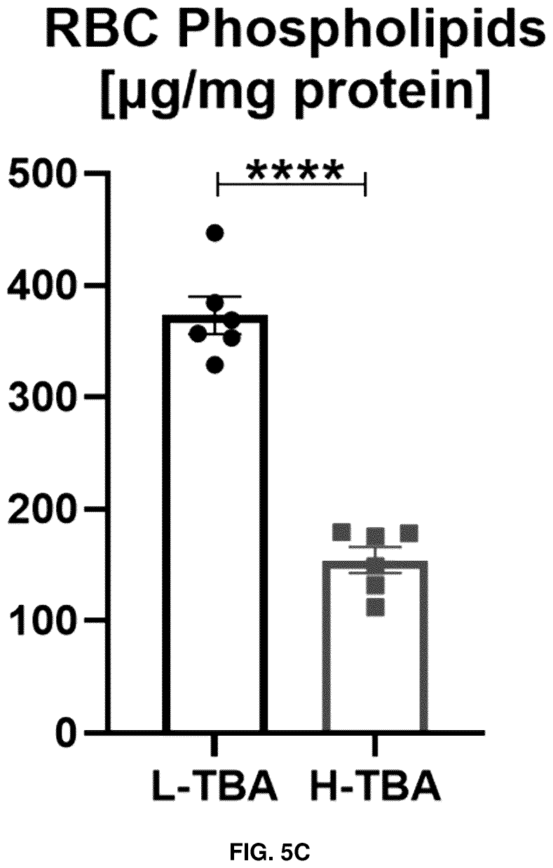

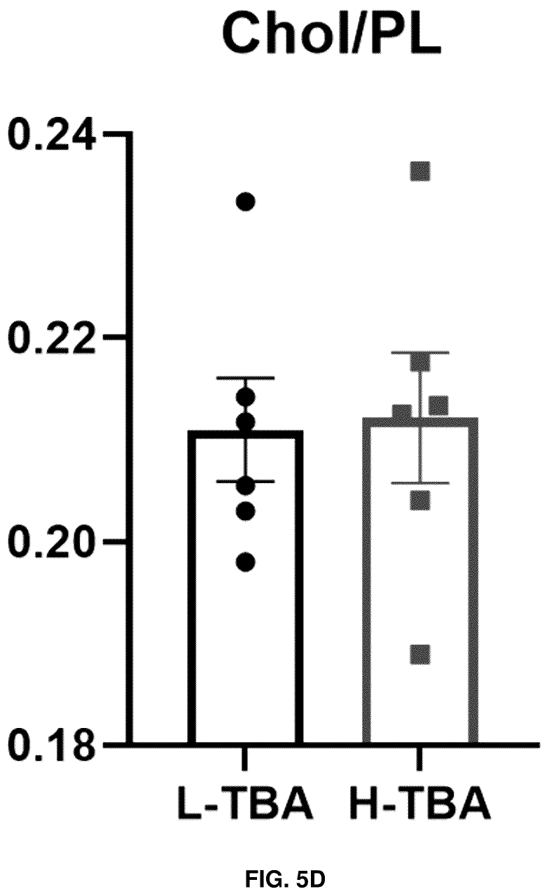

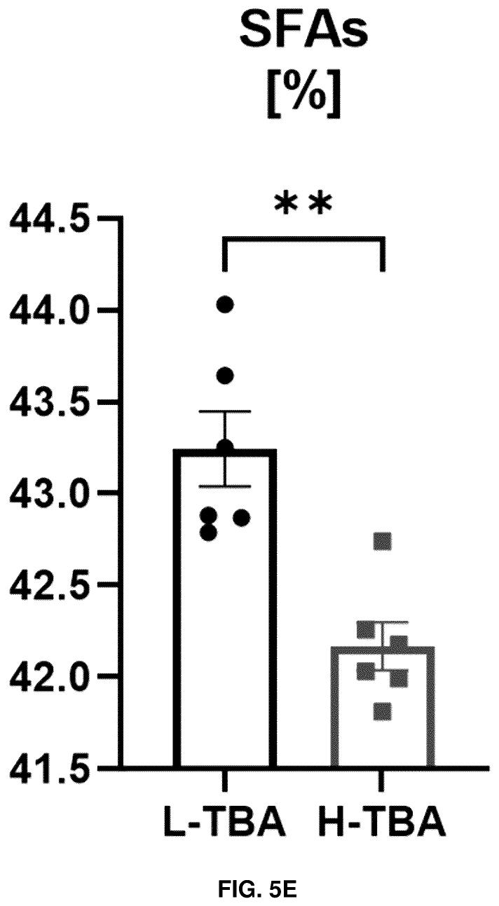

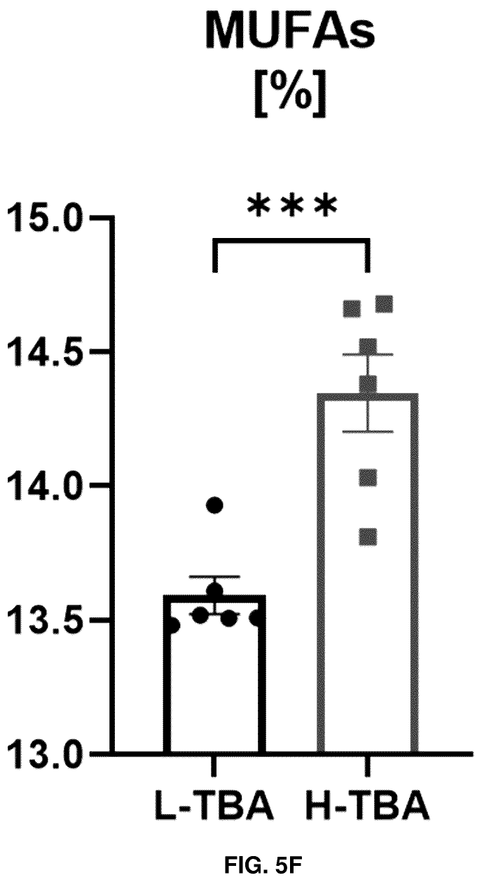

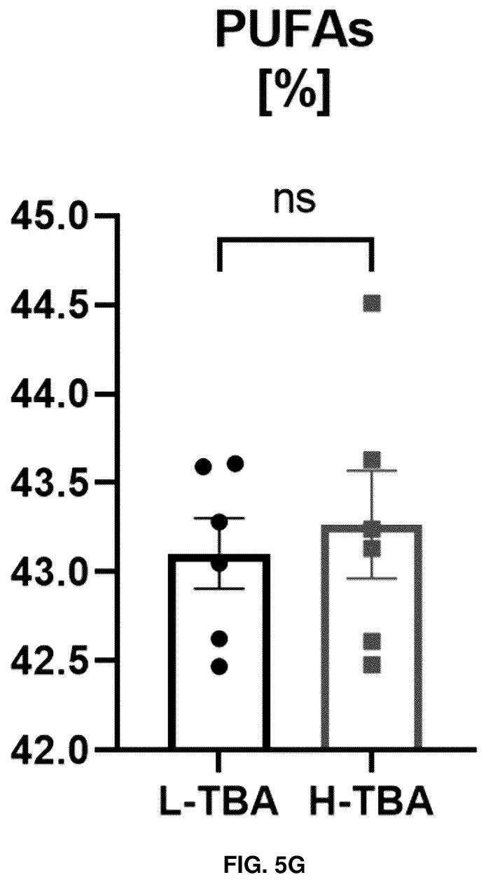

Bile acid-induced membrane damage has conventionally been thought to be due to depletion of phospholipids, and cholesterol enrichment in the membrane has been believed to protect against hemolysis caused by bile acids. The present examples demonstrate H-TBA mice exhibit systemic hypocholesterolemia (FIG. 5A). As such, whether reduced supply of cholesterol for RBC membrane biogenesis may be involved in driving the hemolysis resistance phenotype in H-TBA mice was investigated. Intriguingly, no differences in the amount of cholesterol between L-TBA and H-TBA RBC membranes were found (FIG. 5B). However, there was still a near 60% reduction in RBC phospholipid content in H-TBA RBCs that would come from continual bile acid exposure (FIG. 5C). The similar cholesterol-to-phospholipid ratios between the groups (FIG. 5D) indicates that other membrane components, besides intact cholesterol, are contributing to the hemolysis resistance in H-TBA mice.

Membrane fluidity and fragility has been correlated to the ratio of saturated (SFA), monounsaturated (MUFA), and polyunsaturated fatty acids (PUFA). For instance, increased unsaturated fatty acid quantities enhanced membrane fluidity. When analyzing the RBC phospholipid fatty acid composition in H-TBA mice, a marked reduction (˜2.5%) in SFA was observed (FIG. 5E), but a considerable increase (˜5.3%) in RBC phospholipid MUFA levels was found (FIG. 5F). There were no major differences in the RBC membrane PUFA content (FIG. 5G). These results indicate that increased MUFA promotes membrane fluidity in H-TBA RBCs. Importantly, increased erythrocyte membrane fluidity has been associated with increased resistance to osmotic lysis, indicating that the altered erythrocyte membrane phospholipid content in H-TBA RBCs is involved in the lysis resistance phenotype.

RBCs from Cholestatic Patients are Resistant to NaCl-Induced Osmotic Hemolysis

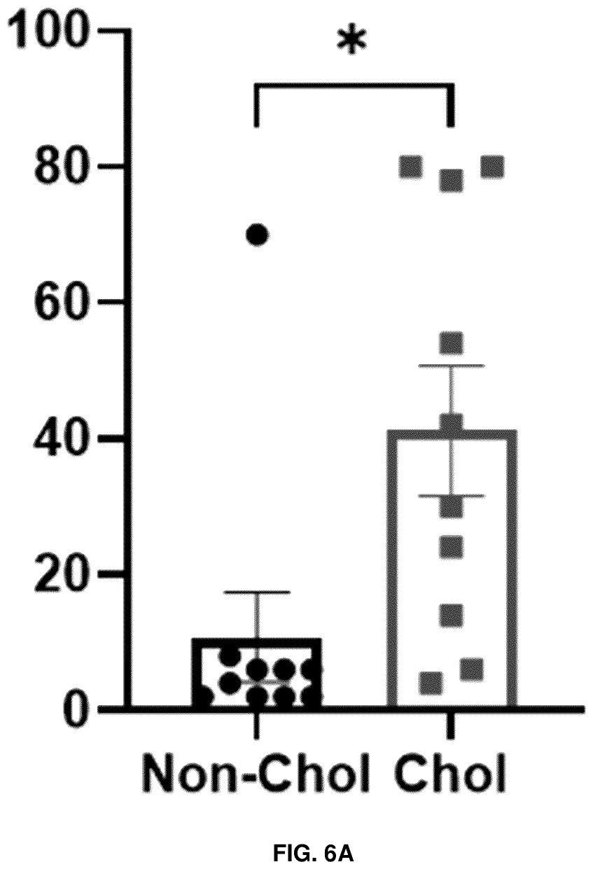

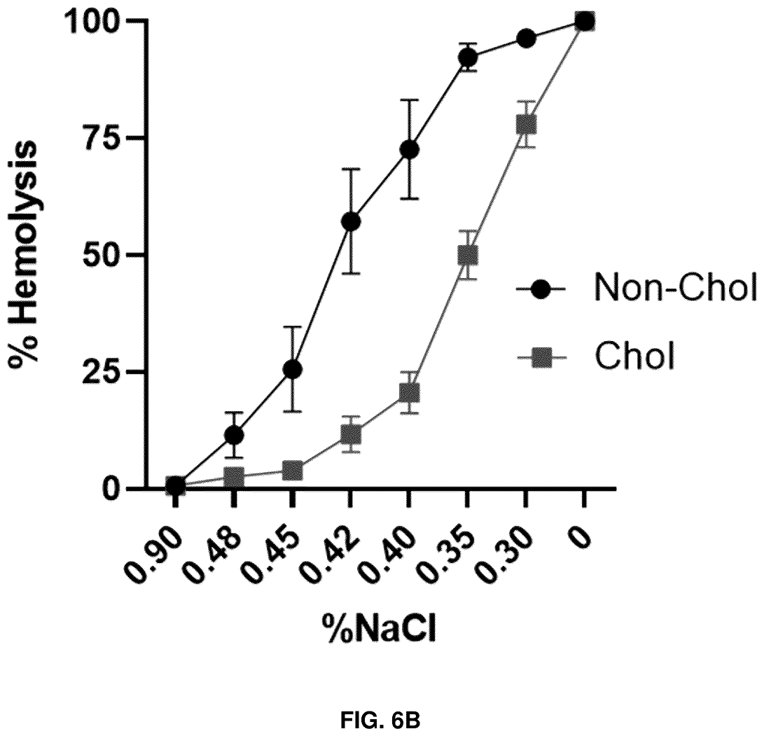

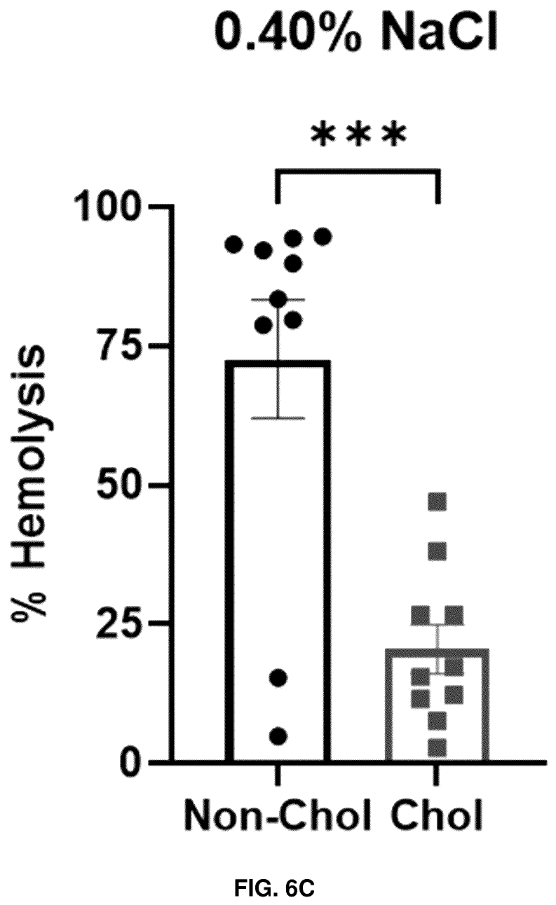

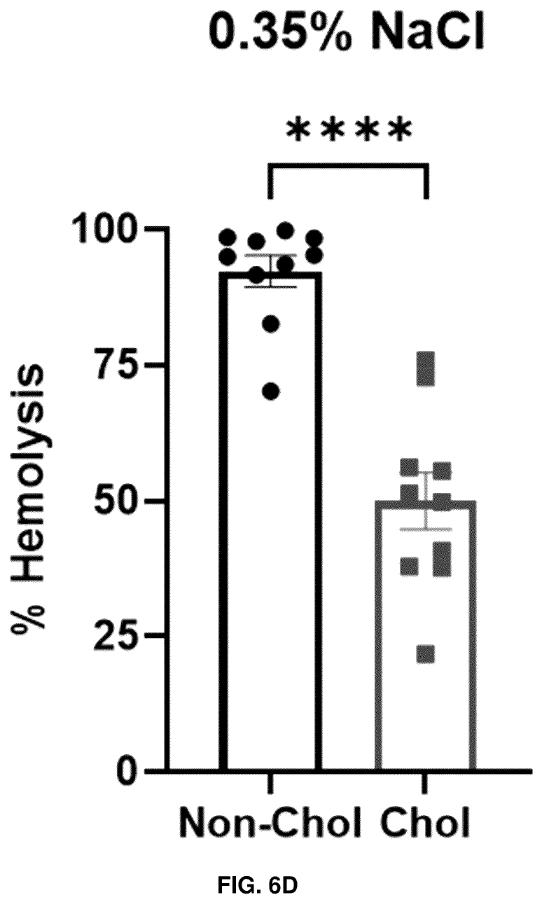

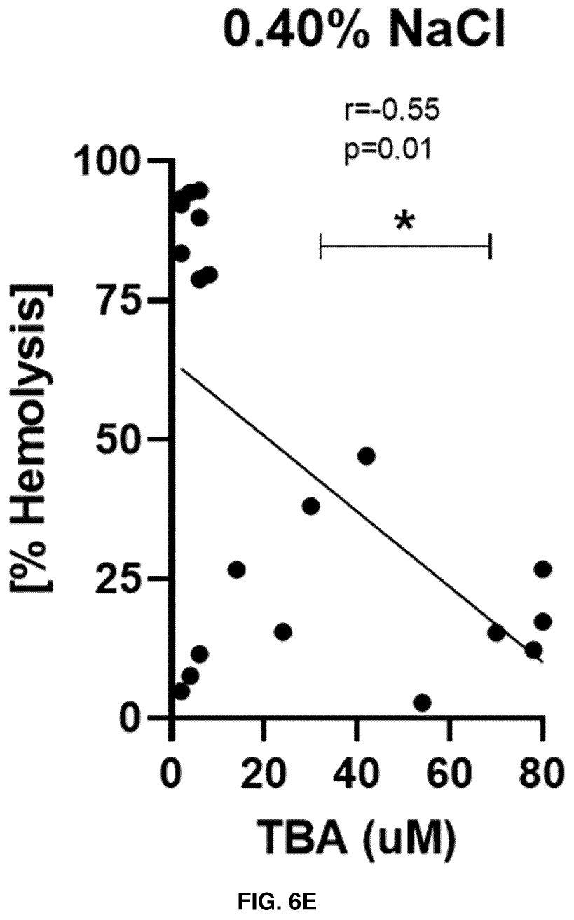

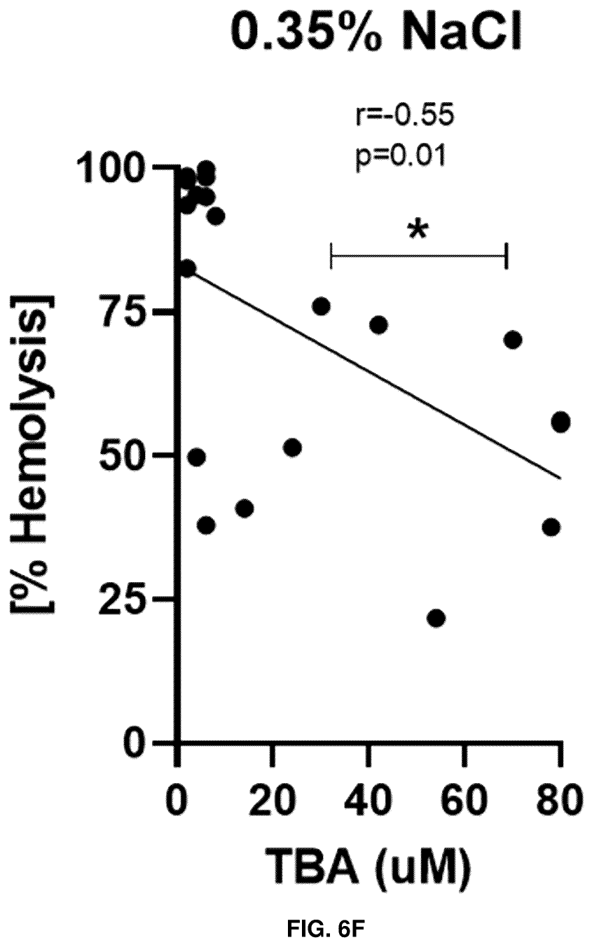

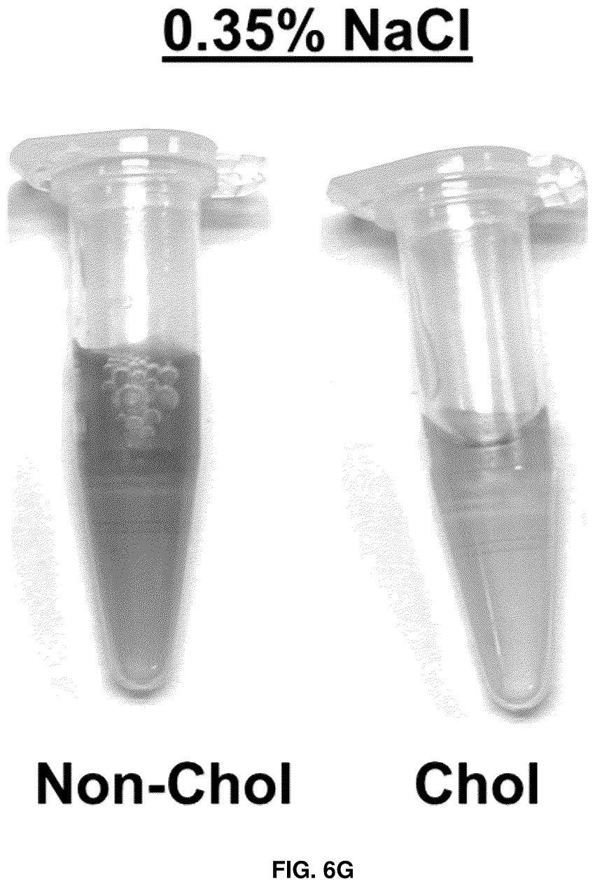

The ascertainment of hemolysis resistance in the H-TBA mice led to the conclusion that osmotic lysis can be used as a biomarker in patients with cholestatic liver disease because these patients exhibit higher circulating bile acids. To test this, whole blood samples from cholestatic patients and healthy controls (no liver disease) were collected at The University of Toledo's Department of Pathology. There was a total of 46 participants for the study (23 cholestatic and 23 non-cholestatic controls), where male patients represented 65.2% (median age: 60 years) and female patients represented 34.8% (Median age: 59 years) of the entire cohort (Table 2). A near 3-fold increase in serum TBA levels in the cholestatic patient group was observed (FIG. 6A), confirming this conclusion. In parallel with the H-TBA mice, RBCs from cholestatic patients were significantly more resistant to osmotic hemolysis (FIG. 6B). Of note, while the most hemolytic resistance in H-TBA mice was in the concentration range of 0.48-0.50% NaCl, cholestatic human RBCs showed greatest resistance between 0.35-0.42% NaCl (FIGS. 6B-6D).

TABLE 2
Characteristics of the cholestatic patient cohort

| | | |
|---|---|---|
| Patients feature | N | % |

| | | | |
|---|---|---|---|
| Age | <60 years | 23 | 50 |
| | >60 years | 23 | 50 |
| | Total | 46 | 100 |
| Sex | Male | 30 | 65 |
| | Female | 16 | 35 |
| Cholestasis? | Yes | 23 | 50 |
| | No | 23 | 50 |
| Cause of | Obstructive | 8 | 35 |
| cholestasis | jaundice | | |
| | Cirrhosis | 7 | 30 |
| | Cancer | 2 | 9 |
| | Unknown | 3 | 13 |
| | etiology | | |
| | Other | 3 | 13 |

Hemolytic anemia is strongly associated with cholestasis. Applying the same hematocrit normalization countermeasures that were done with H-TBA mice, hemolysis was measured using 50% more blood from the cholestasis samples, and significant lysis resistance was still observed (FIGS. 2B-2C). Importantly, as in the mouse model, TBA and hemolysis were observed to be significantly inversely correlated (FIGS. 6E-6F), and the supernatant color could distinguish cholestatic and healthy blood samples when conducting hemolysis in a blinded fashion (FIG. 6G). Collectively, these data confirm that the RBC hemolysis resistance in mice and humans is driven by continual bile acid exposure rather than low RBC quantity from anemia, and that osmotic lysis is useful in clinical practice to screen for cholestatic liver disease.

RBCs from H-TBA Mice and Cholestatic Patients are Resistant to KCl-Induced Osmotic Hemolysis

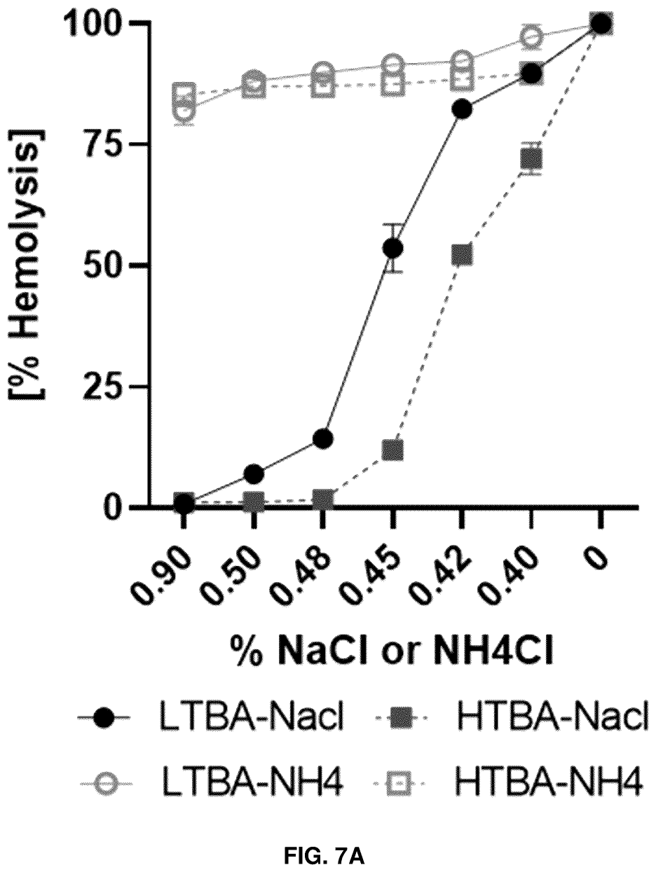

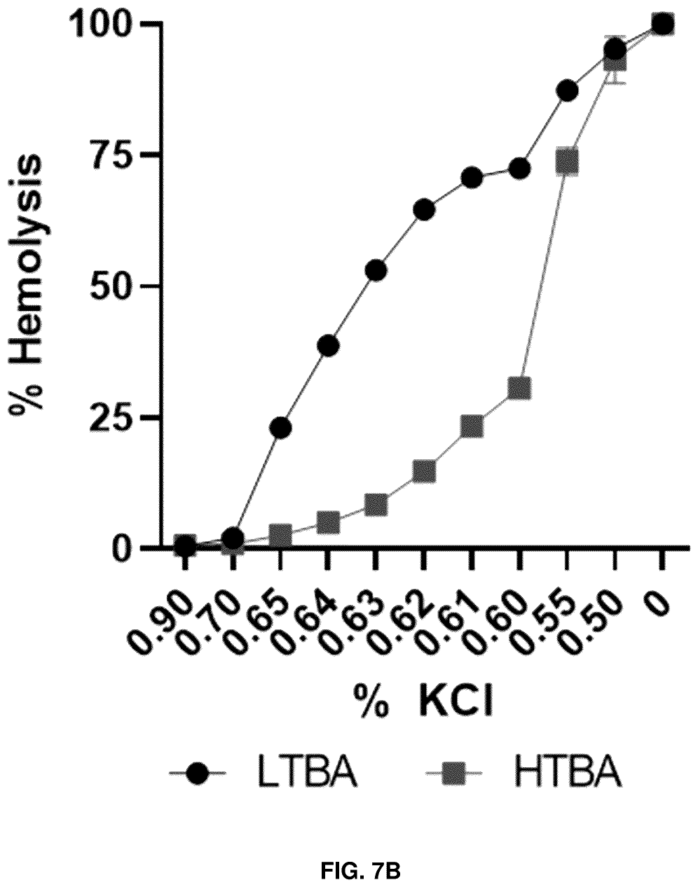

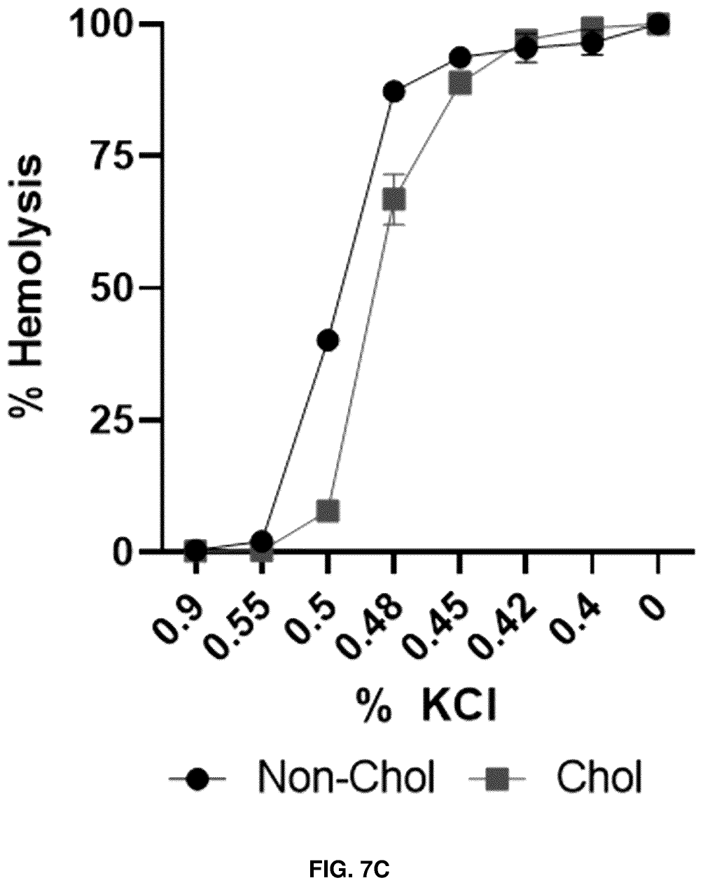

There are other available ionic solutions that are known to induce RBC lysis. Ammonium chloride (NH₄Cl), for instance, is a common active ingredient in commercially available RBC lysis buffers. Interestingly, H-TBA RBCs were not protected from NH₄Cl-induced hemolysis when using the same concentration range as NaCl (FIG. 7A), demonstrating an approach to cause complete RBC lysis independent of TBA status. Potassium chloride (KCl), which is another, but less employed, lysis agent was also tested. Intriguingly, it was observed that H-TBA mice were resistant to KCl-induced lysis at a concentration range of 0.55-0.65% KCl (FIG. 7B). To a lesser extent, RBCs from human cholestatic patients were also resistant to KCl-induced hemolysis, but at a concentration range of 0.45-0.50% KCl (FIG. 7C). These results demonstrate that NaCl and KCl are near equivalent for showing hemolysis resistance in H-TBA mice, but NaCl better illustrates cholestatic patient RBCs protection against hemolysis compared to KCl. Despite NaCl, KCl, and NH₄Cl compounds being ionic salts, it is still unclear why there are distinct differences in osmotic shock responses between RBCs incubated in either of these solutions.

FIG. 8 shows photographs of the serum samples collected from two of the human subjects, referred to as Sample A and Sample B. In the image on the left in FIG. 8, a significant difference in opacity between Sample A and Sample B is seen between concentrations of 0.3% and 0.45%. More opacity shows less lysis has occurred. The photograph in the center of FIG. 8 shows the samples after 30 minutes of incubation plus centrifugation. An increased pellet size in Sample A compared to Sample B is seen after centrifugation. The image on the right in FIG. 8 shows cell-free supernatants from the samples. In this image, Sample A is seen to have significantly less red color in the supernatant. Sample A was cholestatic, and Sample B was not.

### DISCUSSION

The erythrocyte membrane is a two-dimensional structure with a cytoskeleton connected to a lipid bilayer. The structure of the bilayer has equivalent protein and lipid content, where the membrane lipids are primarily phospholipids and neutral lipids with mostly unesterified cholesterol intercalated between the phospholipid molecules. The mechanical and biological properties of the RBC membrane have been previously studied. A submembrane of protein networks, for instance, was found responsible for the viscoelastic property of RBC membranes to resist stretching and deformation when strained. Erythrocyte membrane fluidity was found to be dictated by the relative amounts of cholesterol and phospholipids, where an alteration in its ratio could result in detrimental morphological changes that decrease the RBC life span. As such, an altered blood rheological pattern due to unfavorable lipid composition and structural changes in the RBC membrane may contribute to the development of several diseases, such as diabetes mellitus.

The pathological potential from altered lipid composition and fluidity in the erythrocyte membrane has launched investigations to what could be the causative agents. Several candidates include mechanical sheer stress and toxic radicals from oxidative stress that are common in hereditary RBC membrane disorders. Another proposed major toxin to the RBC membrane is bile acids because their hydrophobicity and detergent-like property allows for membrane penetration and lipid depletion, respectively. In vitro studies with intact cells and RBC membranes have found that treatment with either unconjugated or conjugated cholate (primary bile acid) gave lipid-depleted residues which were analogous to cells administered the commercially available detergent Triton X-100. A depression of lipids and, therefore, impaired membrane fluidity, can result in hemolysis of RBCs. Interestingly, introducing bile acids to human erythrocytes via bile originated from different mammals (i.e., rabbit, guinea pig, pig, ox, or sheep) strongly induced hemolysis, demonstrating the role of bile acids on RBC integrity is irrespective of species. Altogether, the current consensus has been that bile acids at high concentrations are cytotoxic to the RBC membrane.

In the present examples, whether a murine model of cholemia (i.e., high systemic bile acid levels) could recapitulate the prior in vitro observations was investigated. It was discovered that ˜10% of WT C57BL/6 mice develop spontaneous congenital portosystemic shunt caused by an abnormal vessel that allows for portal vein blood to bypass the liver and enter directly into systemic circulation. Indeed, these mice naturally develop cholemia (H-TBA≥40 μM) and their low TBA (L-TBA) WT counterpart, which averages 10 M, matches the upper TBA value for healthy human subjects. The mouse model provided an opportunity to see how bile acids impact RBC physiology without the necessity for surgical, dietary, or genetic approaches that could produce undesired off-target, confounding effects.

The findings in these examples illustrate an intriguing exception to the bile acid toxicity convention where RBCs from H-TBA mice were surprisingly resistant, rather than severely susceptible, to osmotic hemolysis induced by either NaCl or KCl. In fact, the degree of susceptibility versus resistance to hemolysis directly correlated to the TBA value, where 1-5 μM (normal TBA levels) showed the most lysis and 90-100 μM (toxic TBA levels) showed the most resistance in L-TBA and H-TBA mice, respectively. The human cohort with cholestasis patients confirmed that cholemia TBA values provided protection against RBC hemolysis, and this supports an observation that >30% of RBCs in patients with extrahepatic cholestasis have increased resistance to hemolysis. These opposing results from the mouse model and human cohort compared to prior in vitro studies are from the different bile acid concentrations applied in the hemolysis assay. The previous in vitro results found bile acids induced hemolysis when supplemented at a range from 5-20 mM, which is a baseline value for hepatic bile acid levels. The present examples focused on systemic bile acid levels at normal (1-10 μM TBA) and toxic (≥40 μM TBA) concentrations, where both the mouse model and human cohort showed protection against hemolysis when TBA was ≥40 μM. The in vitro results of supplementing unconjugated cholic acid at 100 μM substantiates that the systemic, rather than hepatic, bile acid value correlates to hemolysis resistance.

An interesting dynamic was also found, namely, that the phospholipid depletion in erythrocyte membranes from H-TBA mice did not contribute to lysis susceptibility. Without wishing to be bound by theory, it is believed that this is due, at least in part, to H-TBA RBCs having intact cholesterol levels in the membrane, where cholesterol has been previously noted to reduce the lipid-depleting effects from bile acids. While the present examples did not measure for RBC membrane fluidity, bile acids are still penetrating the lipid bilayer and are, perhaps, promoting membrane fluidity through unknown mechanisms. Intestinal bile acids increase biological membrane fluidity, which can lead to enhanced absorption and bioavailability of drug compounds. The present examples indicate that bile acids are removing the saturated fatty acids, which allows for the expansion of monounsaturated fatty acids to increase membrane fluidity. If, in a cholemia or cholestasis setting, bile acids are protecting RBCs from hemolysis, then this is a possible adaption to minimize consequences for patients that already have complications.

Altogether, the findings from these examples underline a highly compelling and reproducible phenotype that exists in both mice and humans. The ability of bile acids to confer resistance to RBC hemolysis should not be overlooked when performing hematological analyses. The mechanism of how bile acids provide RBC protection against osmotic shock is not currently known. For the clinical settings, measuring hemolysis is an inexpensive and accurate method to prescreen for liver disease in tandem with conventionally used liver panel function tests. Overall, these examples demonstrate that bile acids are beneficial for sustaining erythrocyte membrane integrity.

Certain embodiments of the assays and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the assays and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A method for diagnosing cholemia, the method comprising:
obtaining a blood sample from a subject;
adding a salt to the blood sample to form a solution; and
comparing an amount of hemolysis that takes place in the solution to a reference amount of hemolysis in a normal blood sample in a reference solution with the salt to determine whether the subject has cholemia.

2. The method of claim 1, comprising comparing an opacity of the solution to a reference opacity from a normal blood sample in a reference solution with the salt, wherein the opacity of the solution being greater than the reference opacity indicates that the subject has cholemia.

3. The method of claim 2, wherein the salt comprises NaCl.

4. The method of claim 3, wherein the salt is present at a concentration ranging from about 0.30% by weight to about 0.48% by weight.

5. The method of claim 2, wherein the salt comprises KCl.

6. The method of claim 5, wherein the salt is present at a concentration ranging from about 0.55% by weight to about 0.65% by weight.

7. The method of claim 5, wherein the salt is present at a concentration ranging from about 0.45% by weight to about 0.5% by weight.

8. The method of claim 1, comprising comparing a color of a supernatant from the solution following centrifugation to a reference color from a normal blood sample in a reference solution with the salt following centrifugation, wherein the color of the supernatant being less red than the reference color indicates that the subject has cholemia.

9. The method of claim 8, further comprising incubating the solution for a period of time of from about 15 minutes to about 30 minutes before centrifuging the solution.

10. The method of claim 8, wherein the salt comprises NaCl.

11. The method of claim 10, wherein the salt is present at a concentration ranging from about 0.30% by weight to about 0.48% by weight.

12. The method of claim 8, wherein the salt comprises KCl.

13. The method of claim 12, wherein the salt is present at a concentration ranging from about 0.55% by weight to about 0.65% by weight.

14. The method of claim 12, wherein the salt is present at a concentration ranging from about 0.45% by weight to about 0.5% by weight.

15. The method of claim 1, comprising comparing a pellet size of the solution to a reference pellet size of a normal blood sample solution with the salt.

16. The method of claim 1, wherein the subject is a human.

17. An assay for testing for cholemia, the assay comprising:
a test strip configured to receive a blood sample;
a salt solution; and
a reference chart depicting at least one reference color or reference opacity.

18. The assay of claim 17, further comprising a centrifuge.

19. A method for evaluating resistance to lysis, the method comprising either:
(a) observing a difference in opacity between a first blood sample and a second blood sample, and concluding from the difference in opacity that one of the first blood sample or the second blood sample is more resistant to lysis; or
(b) observing a difference in color between a first supernatant from a first blood sample and a second supernatant from a second blood sample, and concluding from the difference in color that one of the first blood sample or the second blood sample is more resistant to lysis.
